(19) United States
(12) Patent Application Publication
(10) Pub. No.: US 2013/0096159 A1
(43) Pub. Date: Apr. 18, 2013

(54) INHIBITORS OF HUMAN 12-LIPOXYGENASE

(71) Applicants: David J. Maloney, Point of Rocks, MD (US); Theodore Holman, Santa Cruz, CA (US); Ajit Jadhav, Chantilly, VA (US); Anton Simeonov, Bethesda, MD (US); Ganesha Rai Bantukallu, Arlington, VA (US); Jerry L. Nadler, Norfolk, VA (US); Michael Holinstat, Ardmore, PA (US)

(72) Inventors: David J. Maloney, Point of Rocks, MD (US); Theodore Holman, Santa Cruz, CA (US); Ajit Jadhav, Chantilly, VA (US); Anton Simeonov, Bethesda, MD (US); Ganesha Rai Bantukallu, Arlington, VA (US); Jerry L. Nadler, Norfolk, VA (US); Michael Holinstat, Ardmore, PA (US)

(73) Assignees: The United States of America, as represented the Secretary Department of Health & Human Service, Bethesda, MD (US); Thomas Jefferson University, Philadelphia, PA (US); Eastern Virginia Medical School, Norfolk, VA (US); The Regents of the University of California, Oakland, CA (US)

(21) Appl. No.: 13/698,340

(22) PCT Filed: May 18, 2011
(86) PCT No.: PCT/US2011/037000

§ 371 Date: Dec. 18, 2012

Related U.S. Application Data
(60) Provisional application No. 61/345,708, filed on May 18, 2010.

(51) Int. Cl. C07D 409/04; C07D 215/28

(52) U.S. Cl. CPC C07D 409/04; C07D 215/28
USPC 514/314; 546/167; 546/175; 514/311

(57) ABSTRACT

Disclosed are inhibitors of human 12-lipoxygenase of Formula (I) or (II), wherein R¹, R², R³, and R⁴ are as defined herein, that are useful in treating or preventing a 12-lipoxygenase mediated disease or disorder, e.g., diabetes. Also disclosed are a composition comprising a pharmaceutically acceptable carrier and at least one inhibitor of the invention, and a method of treating or preventing such disease or disorder in a mammal.

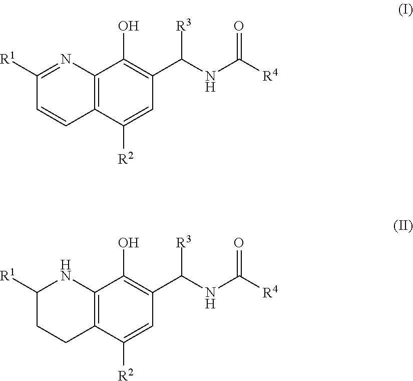

### CROSS-REFERENCE TO A RELATED APPLICATION

This patent application claims the benefit of U.S. Provisional Patent Application No. 61/345,708, filed May 18, 2010, which is incorporated by reference.

### BACKGROUND OF THE INVENTION

Lipoxygenases are a class of non-heme iron-containing enzymes found in plants and animals which catalyze the oxidation of polyunsaturated fatty acids, including those found in lipoproteins, to hydroperoxy derivatives. In humans, there are genes coding for the following lipoxygenases: e-LOX-3 (epidermis-type lipoxygenase 3), 5-LO (5-lipoxygenase), 12-LO (12-lipoxygenase), 12(R)-LOX (12(R)-lipoxygenase), 15-LO-1 (reticulocyte type-15-lipoxygenase-1), and 15-LO-2 (epithelial-type 15-lipoxygenase-2). The lipoxygenases are named according to the specificity of the position of oxidation on arachidonic acid. 12-LO and 15-LO respectively convert arachidonic acid to 12(S)-hydroxyperoxy-5,8,10,14(Z,Z,E,Z)eicosatetraenoic acid (12(S)-HPETE) and 15(S)-hydroxyperoxy-5,8,10,14(Z,Z,E,Z)eicosatetraenoic acid (15(S)-HPETE). Biochemical reduction of 12(S)-HPETE and 15(S)-HPETE respectively leads to the formation of 12(S)-HETE (12-(S)-hydroxy-eicosatetraenoic acid) and 15(S)-HETE (15-(S)-hydroxy-eicosatetraenoic acid) which is the precursor of a class of compounds known as lipoxins.

The 12-lipoxygenase enzyme is found in human monocytes, aortic vascular sooth muscle and endothelial cells, cardiac myocytes, skeletal muscle, the kidney, breast cancer cells, and beta cells of pancreatic islets. Enhanced expression of 12-lipoxygenase is thought to promote cell adhesion, and thus can lead to increased ability of platelets to form large clots in response to vascular injury. Cytokine-induced destruction of pancreatic beta cells seen in type 1 diabetes and islet graft rejection involves multiple intracellular signaling pathways involving products formed by 12-lipoxygenase that directly or indirectly lead to inflammatory damage or programmed cell death. Inflammation also is an important pathological process leading to beta cell dysfunction and death in type 2 diabetes. It is known that inflammatory cytokines rapidly activate 12-LO, and that 12-LO products inhibit insulin secretion, reduce metabolic activity, and induce cell death in human islets. In addition, 12-LO activation is thought to be an important local pathway mediating beta cell dysfunction or reduced beta cell mass in diabetes; Ma et al., J. Clin. Endocrinol. Metab., February 2010, 95(2): 887-893. Furthermore, it is known that products of 12-LO, such as 12-HETE, contribute to platelet-mediated clot formation caused by diabetes and/or cardiovascular disease.

In view of the foregoing, there is a desire to provide new inhibitors of 12-lipoxygenase.

### BRIEF SUMMARY OF THE INVENTION

The invention provides compounds that are potent and selective inhibitors of 12-lipoxygenase. In addition, the present invention provides compositions comprising these compounds and methods of using these compounds as therapeutic agents in the treatment of 12-lipoxyganse mediated diseases or disorders, in particular, in the treatment of diabetes and in the prevention of platelet-mediated clot formation caused by cardiovascular disease.

The invention provides a compound of Formula (I) or Formula (II):

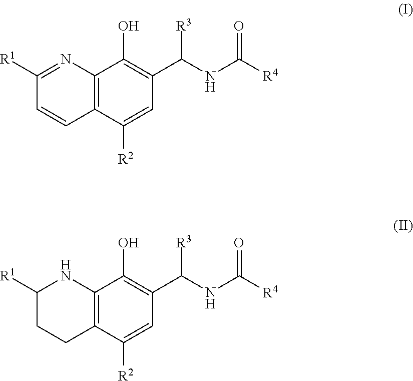

wherein R¹ and R² are independently selected from the group consisting of hydrogen, alkyl, alkenyl, alkynyl, aryl, heterocyclyl, heteroaryl, nitro, fluoro, bromo, chloro, and iodo,

R³ is selected from the group consisting of isoalkyl, cycloalkyl, aryl, heterocyclyl, and heteroaryl, each optionally substituted with one or more substituents selected from the group consisting of halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, heteroaryl, —NO₂, —OH, —OR⁵, —SH, —SR⁵, —SOR⁵, —SO₂R⁵, —COR⁵, —COOH, —COOR⁵, —CONHR⁵, and —CONR⁵R⁶,

R⁴ is selected from the group consisting of hydrogen and alkyl, wherein alkyl is optionally substituted with halo, —NO₂, —OH, —OR⁵, —SH, —SR⁵, —SOR⁵, —SO₂R⁵, —COR⁵, —COOH, —COOR⁵, —CONHR⁵, and —CONR⁵R⁶, and

R⁵ and R⁶ are independently selected from the group consisting of C₁-C₆ alkyl, C₂-C₆ alkenyl, C₂-C₆ alkynyl, C₃-C₈ cycloalkyl, and C₃-C₈ cycloalkenyl,

or a pharmaceutically acceptable salt thereof.

The invention also provides a pharmaceutical composition comprising a compound or salt of the invention and a pharmaceutically acceptable carrier.

The invention further provides a method for treating a 12-lipoxygenase mediated disorder, for example, diabetes, cardiovascular disease, and thrombosis, in a mammal in need thereof, comprising administering a therapeutically effective amount of a compound of the invention or a salt thereof.

Embodiments of the present invention advantageously exhibit high selectivity for 12hLO as compared to 5hLO, 15hLO-1, and 15hLO-2. In addition, embodiments of the present invention exhibit acceptable kinetic aqueous solubility, good cell permeability, and excellent stability in buffer and mouse plasma.

### BRIEF DESCRIPTION OF THE SEVERAL VIEWS OF THE DRAWING(S)

FIG. 1 illustrates a synthetic scheme to prepare compounds of Formula (I) in accordance with an embodiment of the invention.

FIG. 2 illustrates the effect on platelet aggregation in response to stimulation by thrombin (FIG. 2A), arachidonic acid (FIG. 2B), PAR1-AP (FIG. 2C), collagen (FIG. 2D), PAR4-AP (FIG. 2E), and ADP (FIG. 2F) exhibited by a compound in accordance with an embodiment of the invention.

FIG. 3 illustrates the effect on dense granule secretion in response to stimulation by thrombin (FIG. 3A), arachidonic acid (FIG. 3B), PAR1-AP (FIG. 3C), collagen (FIG. 3D), PAR4-AP (FIG. 3E), and ADP (FIG. 3F) exhibited by a compound in accordance with an embodiment of the invention.

FIG. 4 illustrates the effect on α-granule secretion as measured by the increase in P-selectin on the surface of human platelets in response to agonist stimulation by thrombin (FIG. 4A), PAR4-AP (FIG. 4B), PAR1-AP (FIG. 4C), and ADP (FIG. 4D) exhibited by a compound in accordance with an embodiment of the invention.

FIG. 5 illustrates the effect on α-granule secretion as measured by the activation of integrin αIIbβ3 in human platelets in response to agonist stimulation by thrombin (FIG. 5A), PAR4-AP (FIG. 5B), PAR1-AP (FIG. 5C), and ADP (FIG. 5D) exhibited by a compound in accordance with an embodiment of the invention.

FIG. 6 illustrates the effect of 12(S)-HETE on IL-12p40 mRNA levels in human islets.

FIG. 7 illustrates the effect of 12(S)-HETE on IFN-γ mRNA levels in human islets.

FIG. 8 illustrates the effect on cPLA2 activity by a compound in accordance with an embodiment of the invention.

### DETAILED DESCRIPTION OF THE INVENTION

In accordance with an embodiment, the invention provides a compound of Formula (I) or Formula (II):

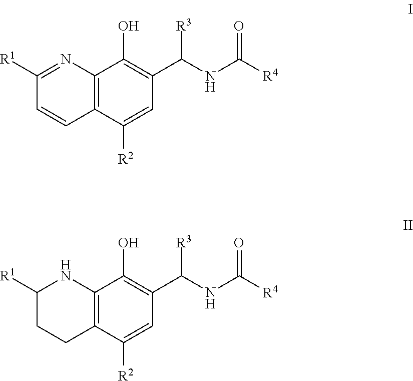

wherein R¹ and R² are independently selected from the group consisting of hydrogen, alkyl, alkenyl, alkynyl, aryl, heterocylyl, heteroaryl, nitro, fluoro, bromo, chloro, and iodo,

R³ is selected from the group consisting of isoalkyl, cycloalkyl, aryl, heterocyclyl, and heteroaryl, each optionally substituted with one or more substituents selected from the group consisting of halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, heteroaryl, —NO₂, —OH, —OR⁵, —SH, —SR⁵, —SOR⁵, —SO₂R⁵, —COR⁵, —COOH, —COOR⁵, —CONHR⁵, and —CONR⁵R⁶,

R⁴ is selected from the group consisting of hydrogen and alkyl, wherein alkyl is optionally substituted with halo, —NO₂, —OH, —OR⁵, —SH, —SR⁵, —SOR⁵, —SO₂R⁵, —COR⁵, —COOH, —COOR⁵, —CONHR⁵, and —CONR⁵R⁶, and

R⁵ and R⁶ are independently selected from the group consisting of C₁-C₆ alkyl, C₂-C₆ alkenyl, C₂-C₆ alkynyl, C₃-C₈ cycloalkyl, and C₃-C₈ cycloalkenyl,

or a pharmaceutically acceptable salt thereof.

Referring now to terminology used generically herein, the term “alkyl” means a straight-chain or branched alkyl substituent containing from, for example, 1 to about 6 carbon atoms, preferably from 1 to about 4 carbon atoms, more preferably from 1 to 2 carbon atoms. Examples of such substituents include methyl, ethyl, propyl, isopropyl, n-butyl, sec-butyl, isobutyl, tert-butyl, pentyl, isoamyl, hexyl, and the like.

The term “alkenyl,” as used herein, means a linear alkenyl substituent containing at least one carbon-carbon double bond and from, for example, 2 to 6 carbon atoms (branched alkenyls are 3 to 6 carbons atoms), preferably from 2 to 5 carbon atoms (branched alkenyls are preferably from 3 to 5 carbon atoms), more preferably from 3 to 4 carbon atoms. Examples of such substituents include vinyl, propenyl, isopropenyl, n-butenyl, sec-butenyl, isobutenyl, tert-butenyl, pentenyl, isopentenyl, hexenyl, and the like.

The term “alkynyl,” as used herein, means a linear alkynyl substituent containing at least one carbon-carbon triple bond and from, for example, 2 to 6 carbon atoms (branched alkynyls are 3 to 6 carbons atoms), preferably from 2 to 5 carbon atoms (branched alkynyls are preferably from 3 to 5 carbon atoms), more preferably from 3 to 4 carbon atoms. Examples of such substituents include ethynyl, propynyl, isopropynyl, n-butynyl, sec-butynyl, isobutynyl, tert-butynyl, pentynyl, isopentynyl, hexynyl, and the like.

The term “cycloalkyl,” as used herein, means a cyclic alkyl substituent containing from, for example, about 3 to about 8 carbon atoms, preferably from about 3 to about 7 carbon atoms, and more preferably from about 3 to about 6 carbon atoms. Examples of such substituents include cyclopropyl, cyclobutyl, cyclopentyl, cyclohexyl, cycloheptyl, cyclooctyl, and the like. The term “cycloalkenyl,” as used herein, means the same as the term “cycloalkyl,” however one or more double bonds are present. Examples of such substituents include cyclopentenyl and cyclohexenyl. The cyclic alkyl groups may be unsubstituted or further substituted with alkyl groups such as methyl groups, ethyl groups, and the like.

The term “heterocyclyl,” as used herein, refers to a monocyclic or bicyclic 5- or 6-membered ring system containing one or more heteroatoms selected from the group consisting of O, N, S, and combinations thereof. The heterocyclyl group can be any suitable heterocyclyl group and can be an aliphatic heterocyclyl group, an aromatic heterocyclyl group, or a combination thereof. The heterocyclyl group can be a monocyclic heterocyclyl group or a bicyclic heterocyclyl group. Suitable bicyclic heterocyclyl groups include monocylic heterocyclyl rings fused to a C₆-C₁₀ aryl ring. When the heterocyclyl group is a bicyclic heterocyclyl group, both ring systems can be aliphatic or aromatic, or one ring system can be aromatic and the other ring system can be aliphatic as in, for example, dihydrobenzofuran. Preferably, the heterocyclyl group is an aromatic heterocyclyl group, which aromatic heterocyclyl group is also referred to as a heteroaryl group. It is understood that a 6-membered heteroaryl group comprises 4n+2π electrons, according to Hückel's Rule, and that a 5-, 7-, and 8-membered heteroaryl group has six electrons provided from a combination of p orbitals and an unshared pair of electrons provided by a heteroatom or heteroatoms which occupy bonding orbitals and constitute an aromatic sextet. Non-limiting examples of suitable heterocyclyl groups include furanyl, thiopheneyl, pyrrolyl, pyrazolyl, imidazolyl, 1,2,3-triazolyl, 1,2,4-triazolyl, isoxazolyl, oxazolyl, isothiazolyl, thiazolyl, pyridinyl, pyrimidinyl, pyrazinyl, triazinyl, benzofuranyl, benzothiopheneyl, indolyl, quinolinyl, isoquinolinyl, benzimidazolyl, benzoxazolinyl, benzothiazolinyl, and quinazolinyl. The heterocyclyl group can be linked at any open position of the heterocyclyl group. For example, the furanyl group can be a furan-2-yl group or a furan-3-yl group, and the thiopheneyl group can be a thiophene-2-yl group or a thiophene-3-yl group. The heterocyclyl group is optionally substituted with 1, 2, 3, 4, or 5 substituents as recited herein, wherein the optional substituent can be present at any open position on the heterocyclyl group.

Whenever a range of the number of atoms in a structure is indicated (e.g., a C₁-C₁₂, C₁-C₈, C₁-C₆, C₁-C₄, or C₂-C₁₂, C₂-C₈, C₂-C₆, C₂-C₄ alkyl, alkenyl, alkynyl, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, alkylamino, etc.) referenced herein encompasses and specifically describes 1, 2, 3, 4, 5, 6, 7, 8, 9, 10, 11, and/or 12 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 1-9 carbon atoms, 1-10 carbon atoms, 1-11 carbon atoms, 1-12 carbon atoms, 2-3 carbon atoms, 2-4 carbon atoms, 2-5 carbon atoms, 2-6 carbon atoms, 2-7 carbon atoms, 2-8 carbon atoms, 2-9 carbon atoms, 2-10 carbon atoms, 2-11 carbon atoms, 2-12 carbon atoms, 3-4 carbon atoms, 3-5 carbon atoms, 3-6 carbon atoms, 3-7 carbon atoms, 3-8 carbon atoms, 3-9 carbon atoms, 3-10 carbon atoms, 3-11 carbon atoms, 3-12 carbon atoms, 4-5 carbon atoms, 4-6 carbon atoms, 4-7 carbon atoms, 4-8 carbon atoms, 4-9 carbon atoms, 4-10 carbon atoms, 4-11 carbon atoms, and/or 4-12 carbon atoms, etc., as appropriate). Similarly, the recitation of a range of 6-10 carbon atoms (e.g., C₆-C₁₀) as used with respect to any chemical group (e.g., aryl) referenced herein encompasses and specifically describes 6, 7, 8, 9, and/or 10 carbon atoms, as appropriate, as well as any sub-range thereof (e.g., 6-10 carbon atoms, 6-9 carbon atoms, 6-8 carbon atoms, 6-7 carbon atoms, 7-10 carbon atoms, 7-9 carbon atoms, 7-8 carbon atoms, 8-10 carbon atoms, and/or 8-9 carbon atoms, etc., as appropriate).

The term “halo” or “halogen,” as used herein, means a substituent selected from Group VIIA, such as, for example, fluorine, bromine, chlorine, and iodine.

The term “aryl” refers to an unsubstituted or substituted aromatic carbocyclic substituent, as commonly understood in the art, and the term “C₆-C₁₀ aryl” includes phenyl and naphthyl. It is understood that the term aryl applies to cyclic substituents that are planar and comprise 4n+2π electrons, according to Hückel's Rule.

In accordance with an embodiment, the compound is of Formula (I).

In accordance with the above embodiment, R¹ is hydrogen.

In any of the above embodiments of Formula (I), R² is selected from the group consisting of nitro, fluoro, chloro, and bromo.

In any of the above embodiments of Formula (I), R³ is selected from the group consisting of isoalkyl or cycloalkyl, heteroaryl, and aryl, each optionally substituted with one or more substituents selected from the group consisting of halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, heteroaryl, —NO₂, —OH, —OR⁵, —SH, —SR⁵, —SOR⁵, —SO₂R⁵, —COR⁵, —COOH, —COOR⁵, —CONHR⁵, and —CONR⁵R⁶.

In any of the above embodiments of Formula (I), R³ is an isoalkyl or cycloalkyl group. In certain preferred embodiments, R³ is an C₃-C₆ isoalkyl group. Examples of suitable C₃-C₆ isoalkyl groups include isopropyl, isobutyl, isopentyl, and isohexyl. The prefix “iso” refers to an alkyl group having a branch point at the carbon atom of the isoalkyl group that is attached to the rest of the molecule. In certain preferred embodiments, R³ is a C₃-C₆ cycloalkyl group. Examples of suitable C₃-C₆ cycloalkyl groups include, cyclopropyl, cyclobutyl, cyclopentyl, and cyclohexyl.

In certain preferred embodiments, the invention provides a compound selected from the group consisting of N-((5-chloro-8-hydroxyquinolin-7-yl)(isopropyl)methyl)propionamide and N-((5-chloro-8-hydroxyquinolin-7-yl)(isopropyl)methyl)acetamide.

In certain preferred embodiments, the invention provides a compound selected from the group consisting of N-((5-chloro-8-hydroxyquinolin-7-yl)(cyclopropyl)methyl)propionamide, and N-((5-chloro-8-hydroxyquinolin-7-yl)(cyclopropyl)methyl)acetamide.

In certain embodiments of Formula (I), R³ is heteroaryl, optionally substituted with one or more substituents selected from the group consisting of halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, heteroaryl, —NO₂, —OH, —OR⁵, —SH, —SRS, —SOR⁵, —SO₂R⁵, —COR⁵, —COOH, —COOR⁵, —CONHR⁵, and —CONR⁵R⁶. In certain preferred embodiments, R³ is furan-2-yl or thiophen-2-yl, and alkylated or halogenated derivatives thereof. Non-limiting examples of alkylated or halogenated derivatives include 5-methylfuran-2-yl, 5-methylthiophen-2-yl, 5-bromofuran-2-yl, and 5-bromothiophen-2-yl. In certain more preferred embodiments, R³ is furan-2-yl or thiophen-2-yl.

In certain preferred embodiments, the invention provides a compound selected from the group consisting of N-((5-nitro-8-hydroxyquinolin-7-yl)(furan-2-yl)methyl)propionamide, N-((5-chloro-8-hydroxyquinolin-7-yl)(furan-2-yl)methyl)propionamide, N-((5-chloro-8-hydroxyquinolin-7-yl)(furan-2-yl)methyl)acetamide, N-((5-bromo-8-hydroxyquinolin-7-yl)(furan-2-yl)methyl)propionamide, N-((5-bromo-8-hydroxyquinolin-7-yl)(furan-2-yl)methyl)acetamide, and N-((5-fluoro-8-hydroxyquinolin-7-yl)(furan-2-yl)methyl)acetamide.

In a particular embodiment, the invention provides a compound which is N-((5-chloro-8-hydroxyquinolin-7-yl)(5-bromofuran-2-yl)methyl)propionamide.

In certain preferred embodiments, the invention provides a compound selected from the group consisting of N-((5-nitro-8-hydroxyquinolin-7-yl)(thiophen-2-yl)methyl)propionamide, N-((5-chloro-8-hydroxyquinolin-7-yl)(thiophen-2-yl)methyl)propionamide, N-((5-chloro-8-hydroxyquinolin-7-yl)(thiophen-2-yl)methyl)acetamide, N-((5-bromo-8-hydroxyquinolin-7-yl)(thiophen-2-yl)methyl)propionamide, N-((5-bromo-8-hydroxyquinolin-7-yl)(thiophen-2-yl)methyl)acetamide, N-((8-hydroxyquinolin-7-yl)(thiophen-2-yl)methyl)propionamide, and N-((5-fluoro-8-hydroxyquinolin-7-yl)(thiophen-2-yl)methyl)acetamide.

In a particular embodiment, the invention provides a compound which is N-((5-chloro-8-hydroxyquinolin-7-yl)(5-methylthiophen-2-yl)methyl)propionamide.

In certain embodiments of Formula (I), R³ is aryl, optionally substituted with one or more substituents selected from the group consisting of halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, heteroaryl, —NO₂, —OH, —OR⁵, —SH, —SR⁵, —SOR⁵, —SO₂R⁵, —COR⁵, —COOH, —COOR⁵, —CONHR⁵, and —CONR⁵R⁶.

In certain preferred embodiments of Formula (I), the invention provides a compound selected from the group consisting of N-((5-chloro-8-hydroxyquinolin-7-yl)(4-methylphenyl)methyl)propionamide or N-((5-chloro-8-hydroxyquinolin-7-yl)(4-fluorophenyl)methyl)propionamide.

In any of the above embodiments, R⁴ is hydrogen or alkyl, wherein alkyl is optionally substituted with halo, —NO₂, —OH, —OR⁵, —SH, —SRS, —SOR⁵, —SO₂R⁵, —COR⁵, —COOH, —COOR⁵, —CONHR⁵, and —CONR⁵R⁶. In certain preferred embodiments, R⁴ is methyl or ethyl.

In accordance with an embodiment, the compound is of Formula (II).

In accordance with an embodiment of Formula (II), R¹ is hydrogen.

In certain embodiments of Formula (II), R² is selected from the group consisting of nitro, fluoro, chloro, and bromo.

In certain embodiments of Formula (II), R³ is selected from the group consisting of isoalkyl or cycloalkyl, heteroaryl, and aryl, each optionally substituted with one or more substituents selected from the group consisting of halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, heteroaryl, —NO₂, —OH, —OR⁵, —SH, —SR⁵, —SOR⁵, —SO₂R⁵, —COR⁵, —COOH, —COOR⁵, —CONHR⁵, and —CONR⁵R⁶.

In certain embodiments of Formula (II), R³ is an isoalkyl or cycloalkyl group. In certain preferred embodiments, R³ is an C₃-C₆ isoalkyl group. Examples of suitable C₃-C₆ isoalkyl groups include isopropyl, isobutyl, isopentyl, and isohexyl. The prefix “iso” is intended to refer to an alkyl group having a branch point at the carbon atom of the isoalkyl group that is attached to the rest of the molecule. In certain preferred embodiments, R³ is a C₃-C₆ cycloalkyl group. Examples of suitable C₃-C₆ cycloalkyl groups include, cyclopropyl, cyclobutyl, cyclopentyl, and cyclohexyl.

In certain embodiments of Formula (II), R³ is heteroaryl, optionally substituted with one or more substituents selected from the group consisting of halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, heteroaryl, —NO₂, —OH, —OR⁵, —SH, —SR⁵, —SOR⁵, —SO₂R⁵, —COR⁵, —COOH, —COOR⁵, —CONHR⁵, and —CONR⁵R⁶. In certain preferred embodiments, R³ is furan-2-yl or thiophen-2-yl, and alkylated or halogenated derivatives thereof. Non-limiting examples of alkylated or halogenated derivatives include 5-methylfuran-2-yl, 5-methylthiophen-2-yl, 5-bromofuran-2-yl, and 5-bromothiophen-2-yl. In more preferred embodiments, R³ is furan-2-yl or thiophen-2-yl.

In a particular embodiment, the invention provides a compound which is the compound is N-((5-nitro-8-hydroxy-1,2,3,4-tetrahydroquinolin-7-yl)(furan-2-yl)methyl)propionamide.

In certain embodiments of Formula (II), R³ is aryl, optionally substituted with one or more substituents selected from the group consisting of halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, heteroaryl, —NO₂, —OH, —OR⁵, —SH, —SR⁵, —SOR⁵, —SO₂R⁵, —COR⁵, —COOH, —COOR⁵, —CONHR⁵, and —CONR⁵R⁶.

In any of the above embodiments, R⁴ is hydrogen or alkyl, wherein alkyl is optionally substituted with halo, —NO₂, —OH, —OR⁵, —SH, —SR⁵, —SOR⁵, —SO₂R⁵, —COR⁵, —COOH, —COOR⁵, —CONHR⁵, and —CONR⁵R⁶. In certain preferred embodiments, R⁴ is methyl or ethyl.

In any of the above embodiments, the compound or salt of Formula (I) or (II) exists in the racemic form, in the form of its pure optical isomers, or in the form of a mixture wherein one isomer is enriched relative to the other. In particular, in accordance with the present invention, when the inventive compounds have a single asymmetric carbon atom, the inventive compounds may exist as racemates, i.e., as mixtures of equal amounts of optical isomers, i.e., equal amounts of two enantiomers. Preferably the compound or salt of Formula (I) or (II) exists in the form of a single enantiomer, and more preferably in the form of a single levorotatory enantiomer. As used herein, “single enantiomer” is intended to mean a compound that comprises more than 50% of a single enantiomer. “Single levorotatory enantiomer,” therefore, means that more than 50% of the levorotatory enantiomer is present along with less than 50% of the dextrorotatory enantiomer (this can also be referred to as a single levorotatory enantiomer), and vice versa (this can also be referred to as a single dextrorotatory enantiomer). As used herein, a levorotatory enantiomer is defined as an enantiomer having a specific rotation at a light wavelength of 589 nm that is negative. By contrast, a dextrorotatory enantiomer is defined as having a specific rotation at a light wavelength of 589 nm that is positive.

Preferably, the single enantiomer comprises at least 75% of a single enantiomer (50% enantiomeric excess) (“e.e.”), more preferably at least 90% of a single enantiomer (80% e.e.), still more preferably at least 95% of a single enantiomer (90% e.e.), even more preferably at least 97.5% of a single enantiomer (95% e.e.), and most preferably at least 99% of a single enantiomer (98% e.e.).

When the compound or salt has more than one chiral center, and can therefore exist as a mixture of diastereomers, preferably the compound or salt exists in the form of a single diastereomer. As used herein, “single diastereomer” is intended to mean a compound that comprises more than 50% of a single diastereomer.

The phrase “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, nonaqueous media such as ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p. 1445, and Journal of Pharmaceutical Science, 66, 2-19 (1977).

Suitable bases include inorganic bases such as alkali and alkaline earth metal bases, e.g., those containing metallic cations such as sodium, potassium, magnesium, calcium and the like. Non-limiting examples of suitable bases include sodium hydroxide, potassium hydroxide, sodium carbonate, and potassium carbonate. Suitable acids include inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, maleic acid, tartaric acid, fatty acids, long chain fatty acids, and the like. Preferred pharmaceutically acceptable salts of inventive compounds having an acidic moiety include sodium and potassium salts. Preferred pharmaceutically acceptable salts of inventive compounds having a basic moiety (e.g., a quinoline group or a dimethylaminoalkyl group) include hydrochloride and hydrobromide salts. The compounds of the present invention containing an acidic or basic moiety are useful in the form of the free base or acid or in the form of a pharmaceutically acceptable salt thereof.

It should be recognized that the particular counterion forming a part of any salt of this invention is usually not of a critical nature, so long as the salt as a whole is pharmacologically acceptable and as long as the counterion does not contribute undesired qualities to the salt as a whole.

It is further understood that the above compounds and salts may form solvates, or exist in a substantially uncomplexed form, such as the anhydrous form. As used herein, the term “solvate” refers to a molecular complex wherein the solvent molecule, such as the crystallizing solvent, is incorporated into the crystal lattice. When the solvent incorporated in the solvate is water, the molecular complex is called a hydrate. Pharmaceutically acceptable solvates include hydrates, alcoholates such as methanolates and ethanolates, acetonitrilates and the like. These compounds can also exist in polymorphic forms.

The present invention is further directed to a pharmaceutical composition comprising a pharmaceutically acceptable carrier and at least one compound or salt described herein.

It is preferred that the pharmaceutically acceptable carrier be one that is chemically inert to the active compounds and one that has no detrimental side effects or toxicity under the conditions of use.

The choice of carrier will be determined in part by the particular compound of the present invention chosen, as well as by the particular method used to administer the composition. Accordingly, there is a wide variety of suitable formulations of the pharmaceutical composition of the present invention. The following formulations for oral, aerosol, nasal, pulmonary, parenteral, subcutaneous, intravenous, intraarterial, intramuscular, intraperitoneal, intrathecal, intratumoral, topical, rectal, and vaginal administration are merely exemplary and are in no way limiting.

The pharmaceutical composition can be administered parenterally, e.g., intravenously, intraarterially, subcutaneously, intradermally, or intramuscularly. Thus, the invention provides compositions for parenteral administration that comprise a solution or suspension of the inventive compound or salt dissolved or suspended in an acceptable carrier suitable for parenteral administration, including aqueous and non-aqueous isotonic sterile injection solutions.

Overall, the requirements for effective pharmaceutical carriers for parenteral compositions are well known to those of ordinary skill in the art. See, e.g., Banker and Chalmers, eds., Pharmaceutics and Pharmacy Practice, J. B. Lippincott Company, Philadelphia, pp. 238-250 (1982), and Toissel, ASHP Handbook on Injectable Drugs, 4th ed., pp. 622-630 (1986). Such solutions can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound or salt of the present invention may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-beta-aminopropionates, and 2-alkyl-imidazoline quaternary ammonium salts, and (e) mixtures thereof.

The parenteral formulations can contain preservatives and buffers. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Topical formulations, including those that are useful for transdermal drug release, are well-known to those of skill in the art and are suitable in the context of the invention for application to skin. Topically applied compositions are generally in the form of liquids, creams, pastes, lotions and gels. Topical administration includes application to the oral mucosa, which includes the oral cavity, oral epithelium, palate, gingival, and the nasal mucosa. In some embodiments, the composition contains at least one active component and a suitable vehicle or carrier. It may also contain other components, such as an anti-irritant. The carrier can be a liquid, solid or semi-solid. In embodiments, the composition is an aqueous solution. Alternatively, the composition can be a dispersion, emulsion, gel, lotion or cream vehicle for the various components. In one embodiment, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In embodiments of the invention, the vehicle for topical application to the skin can include water, buffered solutions, various alcohols, glycols such as glycerin, lipid materials such as fatty acids, mineral oils, phosphoglycerides, collagen, gelatin and silicone based materials.

Formulations suitable for oral administration can consist of (a) liquid solutions, such as a therapeutically effective amount of the inventive compound dissolved in diluents, such as water, saline, or orange juice, (b) capsules, sachets, tablets, lozenges, and troches, each containing a predetermined amount of the active ingredient, as solids or granules, (c) powders, (d) suspensions in an appropriate liquid, and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The compound or salt of the present invention, alone or in combination with other suitable components, can be made into aerosol formulations to be administered via inhalation. The compounds are preferably supplied in finely divided form along with a surfactant and propellant. Typical percentages of active compound are 0.01%-20% by weight, preferably 1%-10%. The surfactant must, of course, be nontoxic, and preferably soluble in the propellant. Representative of such surfactants are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25%-5%. The balance of the composition is ordinarily propellant. A carrier can also be included as desired, e.g., lecithin for intranasal delivery. These aerosol formulations can be placed into acceptable pressurized propellants, such as dichlorodifluoromethane, propane, nitrogen, and the like. They also may be formulated as pharmaceuticals for non-pressured preparations, such as in a nebulizer or an atomizer. Such spray formulations may be used to spray mucosa.

Additionally, the compound or salt of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

It will be appreciated by one of ordinary skill in the art that, in addition to the aforedescribed pharmaceutical compositions, the compound or salt of the present invention may be formulated as inclusion complexes, such as cyclodextrin inclusion complexes, or liposomes. Liposomes serve to target the compounds to a particular tissue, such as lymphoid tissue or cancerous hepatic cells. Liposomes can also be used to increase the half-life of the inventive compound. Liposomes useful in the present invention include emulsions, foams, micelles, insoluble monolayers, liquid crystals, phospholipid dispersions, lamellar layers and the like. In these preparations, the active agent to be delivered is incorporated as part of a liposome, alone or in conjunction with a suitable chemotherapeutic agent. Thus, liposomes filled with a desired inventive compound or salt thereof, can be directed to the site of a specific tissue type, hepatic cells, for example, where the liposomes then deliver the selected compositions. Liposomes for use in the invention are formed from standard vesicle-forming lipids, which generally include neutral and negatively charged phospholipids and a sterol, such as cholesterol. The selection of lipids is generally guided by consideration of, for example, liposome size and stability of the liposomes in the blood stream. A variety of methods are available for preparing liposomes, as described in, for example, Szoka et al., Ann. Rev. Biophys. Bioeng., 9, 467 (1980), and U.S. Pat. Nos. 4,235,871, 4,501,728, 4,837,028, and 5,019,369. For targeting to the cells of a particular tissue type, a ligand to be incorporated into the liposome can include, for example, antibodies or fragments thereof specific for cell surface determinants of the targeted tissue type. A liposome suspension containing a compound or salt of the present invention may be administered intravenously, locally, topically, etc. in a dose that varies according to the mode of administration, the agent being delivered, and the stage of disease being treated.

The invention further provides a method for treating or preventing a 12-lipoxygenase mediated disease or disorder. The method comprises administering an effective amount of the compound of the invention to a mammal afflicted therewith. Preferably, the mammal is a human.

The term “mammal” includes, but is not limited to, the order Rodentia, such as mice, and the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simioids (monkeys) or of the order Anthropoids (humans and apes). An especially preferred mammal is the human. Furthermore, the subject can be the unborn offspring of any of the forgoing hosts, especially mammals (e.g., humans), in which case any screening of the subject or cells of the subject, or administration of compounds to the subject or cells of the subject, can be performed in utero.

The 12-lipoxygenase mediated disease or disorder is typically a disease or disorder wherein the production of 12-hydroperoxyeicosatetraenoic acid (12(S)-HPETE) and/or 12-hydroxyeicosatetraenoic acid (12(S)-HETE) is implicated in the development or progression of the disease or disorder. (12(S)-HETE) and/or (12(S)-HPETE) have been implicated in the reduction of insulin secretion and increased cell death in beta cells found in islets, and are thus implicated in the development of both type I and type II diabetes. See, e.g., Ma et al., J. Clin. Endocrinol. Metab., February 2010, 95(2): 887-893. Thus, inhibition of 12-LO is expected to protect beta cells in human islets. Increased expression and activity of 12-lipoxygenase is implicated in the pathogenesis of cardiovascular diseases such as atherosclerosis and diabetic vascular and kidney disease. In addition, 12-lipoxygenase is upregulated in visceral adipocytes by high-fat feeding in mice, thus suggesting a possible mechanism for the development of diabetes in obese individuals having an excessive amount of visceral fat.

In addition, enhanced expression of 12-lipoxygenase is thought to promote cell adhesion, and thus can lead to increased ability of platelets to form large clots in response to vessel injury. Vascular injury is a critical step in the pathogenesis of coronary artery disease. Platelets become activated at sites of vascular injury and secrete α- and dense granule contents. Platelet α-granules are the primary storage organelle for adhesive and proinflammatory molecules, such as P-selectin, CD40L, and RANTES, but platelet dense granules also contain cell-activating molecules such as serotonin, histamine, and adenine nucleotides, that may be considered to be proinflammatory. Local delivery of adhesive and proinflammatory molecules released from platelet granules may contribute to atherosclerosis and neointima formation after injury. Inhibition of 12-lipoxygenase blocks secretion of dense granules and α-granules by platelets. Activation of 12-lipoxygenase is thought to be required for dense granule secretion by platelets. Thus, selective inhibition of 12-lipoxygenase may be of use in the treatment or prevention of vascular disease with reduction in side effects, such as bleeding.

In accordance with an embodiment, the invention provides a method of treating or preventing diabetes comprising administering to a patient in need thereof a therapeutically effective amount of a compound represented by Formula (I) or (II) or a salt thereof.

In accordance with another embodiment, the invention provides a method of treating or preventing thrombosis comprising administering to a patient in need thereof a therapeutically effective amount of a compound represented by Formula (I) or (II) or a salt thereof.

In accordance with another embodiment, the invention provides a method of treating or preventing cardiovascular disease comprising administering to a patient in need thereof a therapeutically effective amount of a compound represented by Formula (I) or (II) or a salt thereof.

In accordance with another embodiment, the invention provides a method for protecting beta cells in a patient afflicted with diabetes comprising administering to a patient in need thereof a therapeutically effective amount of a compound represented by Formula (I) or (II) or a salt thereof.

“Treating” within the context of the present invention, means an alleviation of symptoms associated with a disorder or disease, or halt of further progression or worsening of those symptoms. For example, within the context of treating patients with diabetes, successful treatment may include a reduction in the amount of insulin required to control blood sugar, or a halting in the progression of a disease such as but not limited to subclinical Cushing's syndrome, testosterone deficiency, high blood pressure, elevated cholesterol levels, coronary artery disease, past gestational diabetes, polycystic ovary syndrome, chronic pancreatitis, fatty liver, hemochromatosis, cystic fibrosis, several mitochondrial neuropathies and myopathies, myotonic dystrophy, and Friedreich's ataxia. Within the context of treating patients with cardiovascular disease, successful treatment may include a reduction in clinical markers such as low density lipoprotein (“LDL”) and lipoprotein A, and/or changes in clinical symptoms such as hypertension, tendency towards thrombosis, and the like. In addition, treatment can be performed in conjunction with or following surgical procedures such as coronary artery bypass graft surgery and cardiac percutaneous coronary intervention. Within the context of protection of beta cells, successful treatment may include a change in dosage of insulin needed to control diabetes or a change in clinical symptoms. Treatment may also include administering the pharmaceutical formulations of the present invention in combination with other therapies. For example, the compounds and pharmaceutical formulations of the present invention may be administered on a chronic basis. The compounds of the invention can also be administered in conjunction with other antidiabetes drugs or cardiovascular drugs. Appropriate combinations can be determined by those of skill in the medical arts.

With regard to treating patients with cardiovascular disease, desirably the treatment does not result in bleeding as a result of the treatment. Drugs currently used in the treatment of platelet disorders including clotting, such as clopidogrel and aspirin, have as a main side effect gastrointestinal hemorrhage and cerebral hemorrhage. In addition, it is known that the platelet integrin αIIbβ3 is intimately involved in the occlusive thrombus formation at the site of endothelial damage, such as occurs in acute coronary syndrome and stroke. Inhibition of 12-LO may result in partial blocking of αIIbβ3 activation and may thus mitigate thrombus formation in these events.

“Preventing” within the context of the present invention, refers to a prophylactic treatment of an individual prone or subject to development of a condition, in particular, a disease or disorder responsive to inhibition of 12-lipoxygenase. For example, those of skill in the medical arts may be able to determine, based on clinical symptoms and patient history, a statistical predisposition of a particular individual to the development of the aforesaid disease or disorder. For example, a family history of diabetes and/or cardiovascular disease and/or various lifestyle factors can be used to assess the predisposition of a particular individual to the development of diabetes and cardiovascular disease and thus inform the individual as to the desirability of preventative treatment with a compound or salt of the invention or a medicament formed therefrom. Accordingly, an individual predisposed to the development of a disease or disorder responsive to inhibition of 12-lipoxygenase may be treated with a compound or a composition of the present invention in order to prevent, inhibit, or slow the development of the disease or disorder.

One skilled in the art will appreciate that suitable methods of utilizing a compound and administering it to a human for the treatment or prevention of disease states, in particular, diabetes, cardiovascular disease, and thrombosis, and for the protection of beta cells, which would be useful in the method of the present invention, are available. Although more than one route can be used to administer a particular compound, a particular route can provide a more immediate and more effective reaction than another route. Accordingly, the described methods are merely exemplary and are in no way limiting.

The dose administered to a mammal, particularly, a human, in accordance with the present invention should be sufficient to effect the desired response. Such responses include reversal or prevention of the bad effects of the disease for which treatment is desired or to elicit the desired benefit. One skilled in the art will recognize that dosage will depend upon a variety of factors, including the age, condition, and body weight of the human, as well as the source, particular type of the disease, and extent of the disease in the human. The size of the dose will also be determined by the route, timing and frequency of administration as well as the existence, nature, and extent of any adverse side-effects that might accompany the administration of a particular compound and the desired physiological effect. It will be appreciated by one of skill in the art that various conditions or disease states may require prolonged treatment involving multiple administrations.

Suitable doses and dosage regimens can be determined by conventional range-finding techniques known to those of ordinary skill in the art. Generally, treatment is initiated with smaller dosages that are less than the optimum dose of the compound. Thereafter, the dosage is increased by small increments until the optimum effect under the circumstances is reached. The present inventive method typically will involve the administration of about 0.1 to about 300 mg of one or more of the compounds described above per kg body weight of the mammal.

By way of example and not intending to limit the invention, the dose of the pharmaceutically active agent(s) described herein for methods of preventing diabetes, cardiovascular disease, and thrombosis can be about 0.001 to about 1 mg/kg body weight of the subject being treated per day, for example, about 0.001 mg, 0.002 mg, 0.005 mg, 0.010 mg, 0.015 mg, 0.020 mg, 0.025 mg, 0.050 mg, 0.075 mg, 0.1 mg, 0.15 mg, 0.2 mg, 0.25 mg, 0.5 mg, 0.75 mg, or 1 mg/kg body weight per day. The dose of the pharmaceutically active agent(s) described herein for methods of treating diabetes, cardiovascular disease, and thrombosis can be about 1 to about 1000 mg/kg body weight of the subject being treated per day, for example, about 1 mg, 2 mg, 5 mg, 10 mg, 15 mg, 0.020 mg, 25 mg, 50 mg, 75 mg, 100 mg, 150 mg, 200 mg, 250 mg, 500 mg, 750 mg, or 1000 mg/kg body weight per day.

The invention further provides a use of a compound or salt of the invention in the manufacture of a medicament for treating or preventing a disease selected from the group consisting of diabetes, cardiovascular disease, and thrombosis, and in the protection of beta cells. The medicament typically is a pharmaceutical composition as described herein.

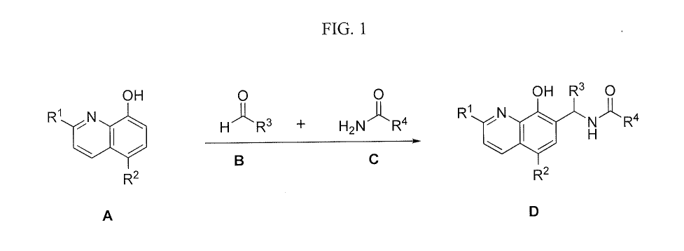

The compounds of the invention can be synthesized by any suitable method, for example, according to the procedure set forth in FIG. 1, wherein R¹, R², R³, and R⁴ are as defined herein. Betti reaction of substituted 8-hydroxyquinolines A with aldehydes B and amides C in the absence of solvent at temperatures of 120° to 150° provided compounds D. Compounds D can be purified via crystallization from suitable solvents and mixtures, thereof, for example, from ethanol-dimethylformamide.

The following examples further illustrate the invention but, of course, should not be construed as in any way limiting its scope.

Unless otherwise stated, all reactions were carried out under an atmosphere of dry argon or nitrogen in dried glassware. Indicated reaction temperatures refer to those of the reaction bath, while room temperature (rt) is noted as 25° C. All solvents were of anhydrous quality purchased from Aldrich Chemical Co. and used as received. Commercially available starting materials and reagents were purchased from Aldrich and were used as received.

¹H- and ¹³C NMR spectra were recorded on a Varian Inova 400 MHz spectrometer. Chemical shifts are reported in ppm with the solvent resonance as the internal standard (CDCl₃ 7.26 ppm, 77.00 ppm, DMSO-d₆ 2.49 ppm, 39.51 ppm for ¹H, ¹³C respectively). Data are reported as follows: chemical shift, multiplicity (s=singlet, d=doublet, t=triplet, q=quartet, br=broad, m=multiplet), coupling constants, and number of protons. Low resolution mass spectra (electrospray ionization) were acquired on an Agilent Technologies 6130 quadrupole spectrometer coupled to the HPLC system. If needed, products were purified via a Waters semi-preparative HPLC equipped with a Phenomenex Luna® C18 reverse phase (5 micron, 30×75 mm) column having a flow rate of 45 mL/min. The mobile phase was a mixture of acetonitrile and H₂O each containing 0.1% trifluoroacetic acid. The mobile phase was a mixture of acetonitrile (0.025% TFA) and H₂O (0.05% TFA), and a temperature was maintained at 50° C.

Samples were analyzed for purity on an Agilent 1200 series LC/MS equipped with a Luna® C18 reverse phase (3 micron, 3×75 mm) column having a flow rate of 0.8-1.0 mL/min over a 7-minute gradient and a 8.5 minute run time. Purity of final compounds was determined to be >95%, using a 3 μL injection with quantitation by AUC at 220 and 254 nm (Agilent Diode Array Detector).

### Example 1

This example demonstrates a general synthesis for the preparation of the compounds of the invention.

A mixture of the quinolin-8-ol (0.5 g, 2.78 mmoles), amide (2.92 mmoles), and aldehyde (3.06 mmoles) were stirred neat at 120-150° C. for 15 minutes. Upon heating, the reaction mixture melted and solid was formed after completion of the reaction. After cooling, the solid product was washed with ethyl acetate and the crude product was crystallized from ethanol-dimethylformamide to provide the purified product.

### Example 2

The following compounds were prepared in accordance with the method described in Example 1.

N-((8-hydroxy-5-nitroquinolin-7-yl)(thiophen-2-yl)methyl)propionamide (1): LC-MS: rt (min)=5.16; ¹H NMR (DMSO-d₆) δ 1.03 (t, J=7.53 Hz, 3H), 2.23 (qd, J=7.56, 3.13 Hz, 2H), 6.81 (dt, J=3.52, 1.17 Hz, 1H), 6.89 (dd, J=8.61, 0.98 Hz, 1H), 6.95 (dd, J=5.09, 3.52 Hz, 1H), 7.43 (dd, J=5.09, 1.17 Hz, 1H), 7.90 (dd, J=8.80, 4.30 Hz, 1H), 8.76 (s, 1H), 9.02 (dd, J=4.11, 1.56 Hz, 1H), 9.09 (d, J=8.80 Hz, 1H), 9.19 (dd, J=8.80, 1.57 Hz, 1H); ¹³C NMR (DMSO-d₆) δ 9.86, 28.38, 45.33, 121.73, 123.84, 125.21, 125.29, 125.35, 126.87, 127.23, 133.05, 134.37, 136.80, 145.17, 149.03, 157.34, 172.29; HRMS (m/z): [M+H]⁺ calcd. for C₁₇H₁₆N₃O₄S, 358.0856. found, 358.0861.

N-((5-chloro-8-hydroxyquinolin-7-yl)(thiophen-2-yl)methyl)propionamide (2): LC-MS: rt (min)=5.6; ¹H NMR (DMSO-d₆) δ 1.03 (t, J=7.6 Hz, 3H), 2.16-2.28 (m, 2H), 6.75-6.78 (m, 1H), 6.89-6.96 (m, 2H), 7.40 (dd, J=5.1 Hz and 1.0 Hz, 1H), 7.74 (dd, J=8.6 Hz and 4.1 Hz, 1H), 7.79 (s, 1H), 8.50 (dd, J=8.5 Hz and 1.5 Hz, 1H), 8.91 (d, J=8.8 Hz, 1H), 8.98 (dd, J=4.1 Hz and 1.4 Hz, 1H) and 10.42 (br, 1H); HRMS (m/z): [M+H]⁺ calcd. for C₁₇H₁₆ClN₂O₂S, 347.0618. found, 347.0621. (−)-2 (e.g. 38) [α]_{D23}=−24 (c=0.6, CHCl₃); (+)-2 (e.g. 39) [α]_{D}²³=+24 (c=0.6, CHCl₃).

N-((5-chloro-8-hydroxyquinolin-7-yl)(thiophen-2-yl)methyl)acetamide (3): LC-MS: rt (min)=5.28; ¹H NMR (DMSO-d₆) δ 1.94 (s, 3H), 6.78 (d, J=3.5 Hz, 1H), 6.89 (d, J=8.8 Hz, 1H), 6.94 (dd, J=5.1 Hz and 3.5 Hz, 1H), 7.41 (dd, J=5.1 Hz and 1.0 Hz, 1H), 7.75 (dd, J=8.5 Hz and 4.2 Hz, 1H), 7.78 (s, 1H), 8.51 (dd, J=8.6 Hz and 1.4 Hz, 1H), 8.96-9.02 (m, 2H) and 10.43 (br, 1H); HRMS (m/z): [M+H]⁺ calcd. for C₁₆H₁₄ClN₂O₂S, 333.0459. found, 333.0460.

N-((5-bromo-8-hydroxyquinolin-7-yl)(thiophen-2-yl)methyl)propionamide (4): LC-MS: rt (min)=5.70; ¹H NMR (DMSO-d₆) δ 1.03 (t, J=7.5 Hz, 3H), 2.16-2.29 (m, 2H), 6.76 (d, J=3.3 Hz, 1H), 6.87-6.96 (m, 2H), 7.40 (dd, J=5.1 Hz and 1.0 Hz, 1H), 7.74 (dd, J=8.5 Hz and 4.2 Hz, 1H), 7.96 (s, 1H), 8.43 (dd, J=8.6 Hz and 1.4 Hz, 1H), 8.86-8.99 (m, 2H) and 10.45 (br, 1H); HRMS (m/z): [M+H]⁺ calcd. for C₁₇H₁₆BrN₂O₂S, 391.0105. found, 391.0108.

N-((5-bromo-8-hydroxyquinolin-7-yl)(thiophen-2-yl)methyl)acetamide (5): LC-MS: rt (min)=5.36; ¹H NMR (DMSO-d₆) δ 1.94 (s, 3H), 6.78 (dt, J=3.5, 1.2 Hz, 1H), 6.89 (dd, J=8.9, 1.1 Hz, 1H), 6.93 (dd, J=5.1, 3.3 Hz, 1H), 7.40 (dd, J=5.1, 1.4 Hz, 1H), 7.74 (dd, J=8.6, 4.1 Hz, 1H), 7.95 (s, 1H), 8.43 (dd, J=8.5, 1.5 Hz, 1H), 8.95 (dd, J=4.1, 1.6 Hz, 1H), 9.00 (d, J=8.8 Hz, 1H), 10.46 (s, 1H); ¹³C NMR (DMSO-d₆) δ 22.55, 45.35, 108.50, 123.41, 124.84, 125.08, 125.68, 126.34, 126.78, 129.38, 134.97, 138.89, 145.91, 149.24, 149.71, 168.39; HRMS (m/z): [M+H]⁺ calcd. for C₁₆H₁₄BrN₂O₂S, 376.9954. found, 376.9956.

N-((5-Fluoro-8-hydroxyquinolin-7-yl)(thiophen-2-yl)methyl)acetamide (7): LC-MS: rt (min)=; ¹H NMR (DMSO-d₆) δ 1.93 (s, 3H), 6.77 (dt, J=3.5, 1.2 Hz, 1H), 6.88-6.95 (m, 2H), 7.40 (dd, J=5.0, 1.3 Hz, 1H), 7.47 (d, J=11.2 Hz, 1H), 7.68 (dd, J=8.5, 4.2 Hz, 1H), 8.44 (dd, J=8.5, 1.7 Hz, 1H), 8.91-8.99 (m, 2H), 10.07 (s, 1H); ¹³C NMR (DMSO-d₆) δ 22.55, 45.49, 104.54, 109.27, 109.48, 117.53, 117.72, 122.27, 123.67, 123.73, 124.86, 125.04, 126.73, 129.13, 129.16, 137.80, 137.83, 145.91, 146.04, 146.08, 148.17, 149.52, 150.60, 168.37; HRMS (m/z): [M+H]⁺ calcd. for C₁₆H₁₄FN₂O₂S, 317.076. found, 317.0761.

N-(furan-2-yl(8-hydroxy-5-nitroquinolin-7-yl)methyl)propionamide (8): LC-MS: rt (min)=4.96; ¹H NMR (DMSO-d₆) δ 1.01 (t, J=7.6 Hz, 3H), 2.16-2.27 (m, 2H), 6.13 (d, J=3.3 Hz, 1H), 6.38 (dd, J=3.1 Hz and 1.8 Hz, 1H), 6.69 (d, J=8.41 Hz, 1H), 7.61 (d, J=1.0 Hz, 1H), 7.90 (dd, J=8.9 Hz and 4.2 Hz, 1H), 8.68 (s, 1H), 8.97 (d, J=8.4 Hz, 1H), 9.01 (dd, J=4.1 Hz and 1.37 Hz, 1H) and 9.17-9.21 (m, 1H); HRMS (m/z): [M+H]⁺ calcd. for C₁₇H₁₆N₃O₅, 342.1084. found, 342.1082.

N-((5-chloro-8-hydroxyquinolin-7-yl)(furan-2-yl)methyl)propionamide (9): LC-MS: rt (min)=5.26; ¹H NMR (DMSO-d₆) δ 1.01 (t, J=7.53 Hz, 3H) 2.20 (qd, J=7.53, 2.64 Hz, 2H) 6.07 (d, J=3.13 Hz, 1H) 6.37 (dd, J=3.03, 1.86 Hz, 1H) 6.72 (d, J=8.61 Hz, 1H) 7.59 (s, 1H) 7.66-7.89 (m, 2H) 8.49 (dd, J=8.51, 1.47 Hz, 1H) 8.80 (d, J=8.80 Hz, 1H) 8.97 (dd, J=4.21, 1.47 Hz, 1H) 10.38 (s, 1H) ¹³C NMR (DMSO-d₆) δ 9.81, 28.28, 43.99, 106.95, 110.39, 118.47, 123.04, 123.07, 125.04, 126.15, 132.50, 138.62, 142.51, 149.16, 149.42, 153.95, 172.21; HRMS (m/z): [M+H]⁺ calcd. for C₁₇H₁₆ClN₂O₃, 331.0844. found, 331.0849.

N-((5-chloro-8-hydroxyquinolin-7-yl)(furan-2-yl)methyl)acetamide (10): LC-MS: rt (min)=4.83; ¹H NMR (DMSO-d₆) δ 1.92 (s, 3H), 6.06-6.09 (m, 1H), 6.37 (dd, J=3.2, 1.9 Hz, 1H), 6.70 (d, J=8.4 Hz, 1H), 7.59 (dd, J=1.9, 0.9 Hz, 1H), 7.71 (s, 1H), 7.74 (dd, J=8.5, 4.2 Hz, 1H), 8.50 (dd, J=8.6, 1.6 Hz, 1H), 8.88 (d, J=8.6 Hz, 1H), 8.97 (dd, J=4.1, 1.6 Hz, 1H), 10.39 (s, 1H); ¹³C NMR (DMSO-d₆) δ 22.51, 44.05, 106.99, 110.39, 118.48, 123.00, 123.05, 125.05, 126.14, 132.52, 138.62, 142.52, 149.19, 149.40, 153.86, 168.47; HRMS (m/z): [M+H]⁺ calcd. for C₁₆H₁₄ClN₂O₃, 317.0687. found, 317.0689.

N-((5-Bromo-8-hydroxyquinolin-7-yl)(furan-2-yl)methyl)propionamide (11): LC-MS: rt (min)=5.39; ¹H NMR (DMSO-d₆) δ 1.01 (t, J=7.6 Hz, 3H), 2.20 (qd, J=7.5, 2.2 Hz, 2H), 6.07 (d, J=3.1 Hz, 1H), 6.37 (dd, J=3.2, 1.9 Hz, 1H), 6.71 (d, J=8.4 Hz, 1H), 7.59 (dd, J=1.8, 0.8 Hz, 1H), 7.73 (dd, J=8.6, 4.1 Hz, 1H), 7.88 (s, 1H), 8.42 (dd, J=8.6, 1.6 Hz, 1H), 8.81 (d, J=8.8 Hz, 1H), 8.94 (dd, J=4.1, 1.6 Hz, 1H), 10.41 (s, 1H); ¹³C NMR (DMSO-d₆) δ 9.81, 28.29, 43.96, 106.94, 108.33, 110.39, 123.38, 123.73, 126.35, 129.63, 134.95, 138.86, 142.51, 149.17, 150.03, 153.96, 172.22; HRMS (m/z): [M+H]⁺ calcd. for C₁₇H₁₆BrN₂O₃, 375.0339. found, 375.0344.

N-((5-Bromo-8-hydroxyquinolin-7-yl)(furan-2-yl)methyl)acetamide (12): LC-MS: rt (min)=5.03; ¹H NMR (DMSO-d₆) δ 1.92 (s, 3H), 6.04-6.11 (m, 1H), 6.37 (dd, J=3.2, 1.9 Hz, 1H), 6.70 (d, J=8.6 Hz, 1H), 7.56-7.62 (m, 1H), 7.73 (dd, J=8.5, 4.2 Hz, 1H), 7.87 (s, 1H), 8.43 (dd, J=8.6, 1.6 Hz, 1H), 8.89 (d, J=8.6 Hz, 1H), 8.94 (dd, J=4.1, 1.6 Hz, 1H), 10.42 (s, 1H); ¹³C NMR (DMSO-d₆) δ 22.51, 43.99, 106.96, 108.33, 110.39, 123.38, 123.65, 126.35, 129.61, 134.96, 138.86, 142.52, 149.18, 150.00, 153.87, 168.47; HRMS (m/z): [M+H]⁺ calcd. for C₁₆H₁₄BrN₂O₃, 361.0182. found, 361.019.

N-((5-Chloro-8-hydroxyquinolin-7-yl)(cyclopropyl)methyl)acetamide (15): LC-MS: rt (min)=4.49; ¹H NMR (DMSO-d₆) δ 0.32-0.40 (m, 3H), 0.43-0.52 (m, 1H), 1.15-1.25 (m, 1H), 1.85 (s, 3H), 4.99 (t, J=8.4 Hz, 1H), 7.70 (dd, J=8.5, 4.2 Hz, 1H), 7.76 (s, 1H), 8.41 (d, J=8.6 Hz, 1H), 8.47 (dd, J=8.4, 1.6 Hz, 1H), 8.95 (dd, J=4.1, 1.6 Hz, 1H), 10.06 (s, 1H); ¹³C NMR (DMSO-d₆) δ 2.38, 3.50, 16.46, 22.65, 49.51, 118.37, 122.67, 124.51, 126.19, 126.38, 132.39, 138.62, 148.78, 148.98, 168.25; HRMS (m/z): [M+H]⁺ calcd. for C₁₅H₁₆ClN₂O₂, 291.0895. found, 291.090.

N-((5-chloro-8-methoxyquinolin-7-yl)(furan-2-yl)methyl)propionamide (33): LC-MS: rt (min)=4.91; ¹H NMR (DMSO-d₆) δ 1.00 (t, J=7.5 Hz, 3H), 2.21 (qd, J=7.5, 5.6 Hz, 2H), 4.06 (s, 3H), 6.16 (d, J=3.1 Hz, 1H), 6.40 (dd, J=3.2, 1.9 Hz, 1H), 6.74 (d, J=8.6 Hz, 1H), 7.61 (d, J=1.8 Hz, 1H), 7.73 (dd, J=8.5, 4.2 Hz, 1H), 7.79 (s, 1H), 8.54 (dd, J=8.6, 1.8 Hz, 1H), 8.90 (d, J=8.6 Hz, 1H), 9.03 (dd, J=4.1, 1.6 Hz, 1H); ¹³C NMR (DMSO-d₆) δ 9.72, 28.24, 44.31, 62.33, 107.29, 110.49, 122.81, 124.76, 125.65, 126.06, 132.65, 132.73, 142.45, 142.77, 150.59, 151.81, 153.51 and 172.31; HRMS (m/z): [M+H]⁺ calcd. for C₁₈H₁₈ClN₂O₃, 345.1. found, 345.1008.

N-((4-chloro-1-hydroxynaphthalen-2-yl)(furan-2-yl)methyl)acetamide (34): LC-MS: rt (min)=5.87; ¹H NMR (DMSO-d₆) δ 1.90 (s, 3H), 6.13 (d, J=3.13 Hz, 1H), 6.39 (dd, J=3.03, 1.86 Hz, 1H), 6.73 (d, J=8.41 Hz, 1H), 7.51-7.58 (m, 1H), 7.58-7.72 (m, 3H), 8.07 (d, J=8.02 Hz, 1H), 8.29 (d, J=8.41 Hz, 1H), 8.94 (d, J=8.41 Hz, 1H), 10.00 (s, 1H); ¹³C NMR (DMSO-d₆) δ 22.43, 44.56, 107.06, 110.37, 121.01, 122.29, 123.06, 123.56, 125.48, 126.09, 126.49, 127.59, 129.94, 142.53, 148.84, 153.91, 168.94; HRMS (m/z): [M+H]⁺ calcd. for C₁₇H₁₅ClNO₃, 316.0735. found, 316.0725.

### Example 3

This example illustrates the functional bioactivity of inventive compounds of Formula I, in accordance with an embodiment, using the human 12-lipoxygenase inhibition (“12hLO”) assay.

The enzyme activity of 12hLO was determined by a direct measurement of product formation by monitoring the absorbance at 234 nm in a 2 mL cuvette. IC₅₀ values of inhibitors were obtained by measuring the enzymatic rate at a variety of concentrations.

For control experiments, 2 mL of substrate buffer (10 M arachidonic acid/25 mM HEPES/0.01% (v/v) Triton X-100, pH 8.0) was aliquoted in a cuvette with a magnetic stir bar. After equilibrium was ensured, an aliquot of inhibitor solvent was added (DMSO), and equilibrium was once again assured. The reaction was started by adding enzyme to the cuvette and the reaction was followed until completed. The inhibition experiments were performed as above, except the actual inhibitory compound was added instead of vehicle. To achieve an IC₅₀, typically 5 concentrations of the inhibitor were studied. If the inhibitor concentration was constant, then five different reaction volumes were used. All experiments were performed in duplicates twice.

Using Kinlab, the largest derivative of the rate at 234 nm is used for the data point in AU/s. Accordingly, the % inhibition is expressed as a ratio of the control to experimental rates (eq. 1):

percent inhibition=[1−(experimental rate)/(control rate)]*100% (eq. 1)

Each percent inhibition data point is plotted as a function of concentration. The plot is then fit to a hyperbolic curve using the equation 2:

([I]*Iₘₐₓ)/([I]+IC₅₀) (eq. 2)

where [I] is the inhibitor concentration, Iₘₐₓ is the maximum percent inhibition and IC₅₀ is the concentration at 50% inhibition. Iₘₐₓ and the IC₅₀ values are extracted from the hyperbolic curve fit. The results are set forth in Table 1.

TABLE 1
12hLO Inhibition

| | | | | | Formula (I) |
|---|---|---|---|---|---|
| 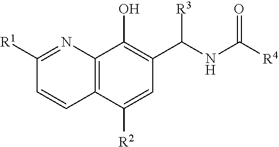 | | | | | |
| | | | | | IC₅₀ (μM) |
| Compound | R¹ | R² | R³ | R⁴ | [± SD μM] |
| 1 | H | NO₂ | thiophen-2-yl | Et | 0.8 [0.2] |
| 2 | H | Cl | thiophen-2-yl | Et | 1.0 [0.3] |
| 3 | H | Cl | thiophen-2-yl | Me | 1.0 [0.1] |
| 4 | H | Br | thiophen-2-yl | Et | 14 [3.0] |
| 5 | H | Br | thiophen-2-yl | Me | 1.0 [0.2] |
| 6 | H | H | thiophen-2-yl | Et | 3.4 [0.6] |
| 7 | H | F | thiophen-2-yl | Me | 2.0 [0.2] |
| 8 | H | NO₂ | furan-2-yl | Et | 1.2 [0.4] |
| 9 | H | Cl | furan-2-yl | Et | 1.0 [0.2] |
| 10 | H | Cl | furan-2-yl | Me | 3.0 [0.5] |
| 11 | H | Br | furan-2-yl | Et | 2.0 [0.5] |
| 12 | H | Br | furan-2-yl | Me | 2.0 [0.3] |
| 13 | H | F | furan-2-yl | Me | 5.0 [1] |
| 14 | H | Cl | cyclopropyl | Et | 1.6 [0.3] |
| 15 | H | Cl | cyclopropyl | Me | 3.0 [0.6] |
| 16 | H | F | cyclopropyl | Me | >150 |
| 17 | H | Cl | isopropyl | Et | 1.2 [0.4] |
| 18 | H | Cl | isopropyl | Me | 2.6 [0.4] |
| 19 | H | F | isopropyl | Me | >150 |
| 20 | H | Cl | Me | Et | >50 |
| 21 | H | Cl | Me | Me | >150 |
| 22 | H | F | Me | Me | >75 |
| 23 | H | Cl | H | Me | >150 |
| 24 | H | Cl | 5-Me-thiophen- | Et | 3.5 [1] |
| | | | 2-yl | | |
| 25 | H | Cl | 5-bromofuran- | Et | <75 |
| | | | 2-yl | | |
| 26 | Cl | Cl | furan-2-yl | Me | >75 |
| 27 | N(Me)₂ | Cl | furan-2-yl | Me | >75 |
| 28 | piperidine | Cl | furan-2-yl | Me | >75 |
| 29 | H | Cl | 4-methylphenyl | Et | >150 |
| 30 | H | Cl | 4-fluorophenyl | Et | >50 |
| 31 | H | Cl | furan-2-yl | Ph | >25 |
| 32 | H | Cl | furan-2-yl | 4-Me—Ph | >25 |

### Example 4

This example illustrates some of the properties of inventive compounds of Formula I, in accordance with an embodiment of the invention.

Compounds 1, 3, 5, 6, 9, 36, and 38 were screened against human 12-lipoxygenase (“12hLO”), human 5-lipoxygenase (“5hLO”), human 15-lipoxygenase-1 (“15hLO-1”), and human 15-lipoxygenase-2 (“15hLO-2”). The IC₅₀ values are set forth in Table 2.

TABLE 2
12hLO, 5hLO, 15hLO-1, and 15hLO-2 Inhibition

IC₅₀ (μM)

| Compound | 12hLO | 5hLO | 15hLO-1 | 15hLO-2 |
|---|---|---|---|---|
| 1 | 0.8 | ND | >25 | ND |
| 3 | 1.0 | >200 | >25 | ND |
| 5 | 1.0 | >500 | >150 | >150 |
| 6 | 3.4 | >150 | >50 | >150 |
| 9 | 1.0 | >150 | >50 | >150 |
| 36 | 0.43 | >250 | >30 | ND |
| 38 | 0.38 | >500 | >50 | ND |

As is apparent from the results set forth in Table 2, compounds 1, 3, 5, 6, 9, 36, and 38 exhibited selectivity for inhibition of human 12-lipoxygenase as compared with inhibition of human 5-lipoxygenase, human 15-lipoxygenase-1 and human 15-lipoxygenase-2.

### Example 5

This example illustrates chiral separation of compounds of Formula (I):

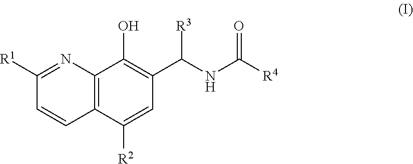

wherein R¹ is H, R² is fluoro, chloro, bromo, or nitro, R³ is furan-2-yl, and R⁴ is methyl or ethyl.

Analytical analysis was performed on a Chiralcel OD column (4.6×150 mm, 5 micron). The mobile phase was 100% methanol at 1.0 mL/min. The sample was detected with a diode array detector (DAD) at 220 nm and 254 nm. Optical rotation was determined with an in-line polarimeter (PDR-Chiral).

Preparative purification was performed on a Chiralcel OD column (2×25 cm, 5 micron). The mobile phase was 100% methanol at 4.5 mL/min. Fraction collection was triggered by UV absorbance (254 nm). The LC system was limited to 100 microliter injections.

### Example 6

This example illustrates chiral separation of compounds of Formula (III):

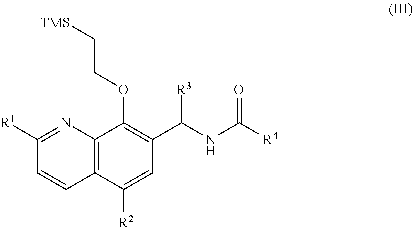

wherein R¹ is H, R² is fluoro, chloro, bromo, or nitro, R³ is furan-2-yl, and R⁴ is methyl or ethyl, which are the 2-trimethylsilylethyl derivatives of compounds of Formula (I).

Compounds of Formula (I) were converted to compounds of Formula (III) using a known method.

Analytical analysis was performed on a Chiralcel IA column (4.6×250 mm, 5 micron). The mobile phase was 60% isopropanol/hexanes at 1.0 mL/min. The sample was detected with a diode array detector (DAD) at 220 nm and 254 nm. Optical rotation was determined with an in-line polarimeter (PDR-Chiral).

Preparative purification was performed on a Chiralcel OD column (2×25 cm, 5 micron). The mobile phase was 60% isopropanol/hexanes, Fraction collection was triggered by UV absorbance (254 nm). The LC system was limited to 100 microliter injections.

### Example 7

This example illustrates the conversion of separated enantiomers of Formula (III) to enantiomers of Formula (I).

Following resolution of enantiomers of Formula (III) by the method described in Example 6, separated enantiomers of Formula (III) were treated with tetra-n-butylammonium fluoride in tetrahydrofuran at room temperature. After work-up of the reaction mixtures, the resulting enantiomers of Formula (I) were isolated via purification by reverse-phase HPLC. The % enantiomeric excess of the enantiomers of Formula (I) were determined using the analytical method described in Example 5.

### Example 8

This example illustrates the 12-lipoxygenase inhibition observed for a racemic mixture of enantiomers and for each of the two resolved enantiomers, in accordance with an embodiment of the invention.

N-((5-bromo-8-hydroxyquinolin-7-yl)(thiophen-2-yl)methyl)acetamide (5) was resolved into its levorotatory enantiomer and its dextrorotatory enantiomer as described in Example 5. Inhibition of 12-lipoxygenase was determined using the method described in Example 3 for racemic 5 (“(±)-5”), levorotatory 5 (36), and dextrotatory 5 (37). The results are set forth in Table 3.

TABLE 3
12hLO Inhibition by Enantiomers

| | IC₅₀ (μM) [(+/−standard |
|---|---|
| Compound | deviation (μM)] |
| (±)-5 (racemate) | 1.0 [0.2] |
| 36 (levorotatory enantiomer) | 0.43 [0.04] |
| 37 (dextrorotatory enantiomer) | >25 |

As is apparent from the results set forth in Table 3, the levorotatory enantiomer 36 of compound 5 was more than 58 times more potent as an inhibitor of 12-lipoxygenase as the dextrorotatory enantiomer 37 of compound 5.

### Example 9

This example illustrates the 12-lipoxygenase inhibition observed for a racemic mixture of enantiomers and for each of the two resolved enantiomers, in accordance with an embodiment of the invention.

N-((5-chloro-8-hydroxyquinolin-7-yl)(thiophen-2-yl)methyl)proprionamide (2) was resolved into its levorotatory enantiomer and its dextrorotatory enantiomer as described in Example 5. Inhibition of 12-lipoxygenase was determined using the method described in Example 3 for racemic 2 (“(±)-2”), levorotatory 2 (38), and dextrotatory 2 (39). The results are set forth in Table 4.

TABLE 4
12hLO Inhibition by Enantiomers

| | IC₅₀ (μM) [(+/−standard |
|---|---|
| Compound | deviation (μM)] |
| (±)-2 (racemate) | 1.0 [0.3] |
| 38 (levorotatory enantiomer) | 0.38 [0.05] |
| 39 (dextrorotatory enantiomer) | >25 |

As is apparent from the results set forth in Table 3, the levorotatory enantiomer of compound 2 was more than 66 times more potent as an inhibitor of 12-lipoxygenase 38 as the dextrorotatory enantiomer 39 of compound 2.

### Example 10

This example describes the separation of human platelets from human blood.

Human platelets were obtained from healthy volunteers from within the Thomas Jefferson University community and the Philadelphia area. These studies were approved by the Thomas Jefferson University Institutional Review Board. Informed consent was obtained from all donors before blood draw. Blood was centrifuged at 200 g for 15 min at room temperature. Platelet-rich plasma was transferred into a conical tube containing a 10% acid citrate dextrose solution (39 mM citric acid, 75 mM sodium citrate, and 135 mM glucose, pH 7.4) and centrifuged at 2000 g for 15 min at room temperature. Platelets were resuspended in Tyrode's buffer (12 mM NaHCO3, 127 mM NaCl, 5 mM KCl, 0.5 mM NaH2PO4, 1 mM MgCl2, 5 mM glucose, and 10 mM HEPES), and the final platelet concentration was adjusted as indicated after counting using a Coulter counter (Beckman Coulter, Fullerton, Calif.). Reported results are the data obtained using platelets from at least three different subjects.

### Example 11

This example demonstrates the effect on platelet aggregation exhibited by a compound in accordance with an embodiment of the invention.

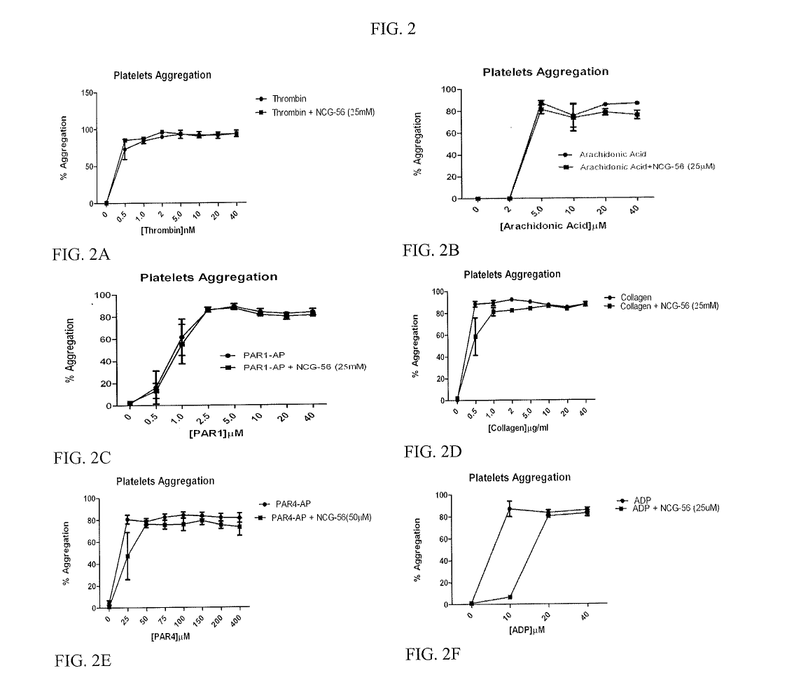

Washed platelets were obtained as described in Example 10. The washed platelets were adjusted to a final concentration of 2×10⁸ platelets/ml. Platelets were pretreated with compound 1 for 10 min and aggregation in response to stimulation by the agonists thrombin, PAR1-AP, PAR4-AP, ADP and collagen at various agonist concentrations was measured via the light transmission aggregometry method using a lumi-aggregometer model 700D (Chrono-log Corp., Havertown, Pa.) with stirring at 1100 rpm at 37° C. The results are depicted in FIG. 2 as dose response curves for platelet aggregation expressed on a percentage basis as a function of agonist concentration, both in the absence and in the presence of compound 1 (identified as NCG-56 in FIG. 2).

As is apparent from the results depicted in FIG. 2, the dose response curves for platelet aggregation expressed on a percentage basis as a function of agonist concentration were substantially unaffected by the presence of compound 1.

### Example 12

This example demonstrates the effect on dense granule secretion in response to agonist stimulation exhibited by a compound in accordance with an embodiment of the invention.

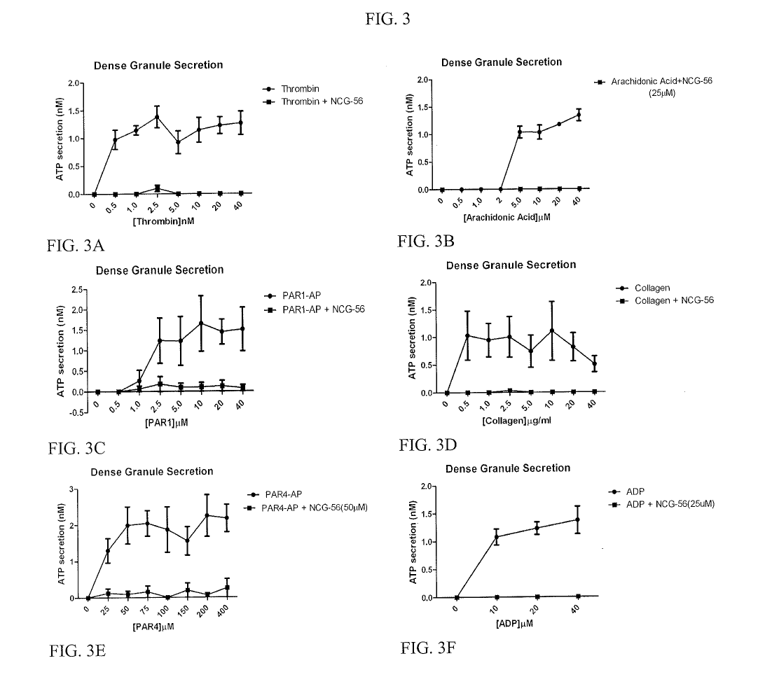

Washed platelets were obtained as described in Example 10. ATP, which is released from platelet dense granules, was used to detect dense granule secretion. 245 μl of washed platelets adjusted to a final concentration of 2×10⁸ platelets/ml were pretreated with inhibitors for 10 minutes. After addition of 5 μl of Chrono-lume reagent (Chrono-log Corp., Havertown, Pa.), ATP release was measured in response to stimulation by the agonists thrombin, PAR1-AP, PAR4-AP, ADP, arachidonic acid, and collagen using a Lumi-aggregometer Model 700D (Chrono-log Corp., Havertown, Pa.) at 37° C. with stirring at 1100 rpm. The results are depicted in FIG. 3 as dose-response curves for ATP secretion as a function of agonist concentration, both in the absence and in the presence of compound 1 (identified as NCG-56 in FIG. 3).

As is apparent from the results depicted in FIG. 3, the presence of compound 1 resulted in substantially complete blockage of ATP secretion induced by the agonists thrombin, PAR1-AP, PAR4-AP, ADP, arachidonic acid, and collagen.

### Example 13

This example demonstrates the effect on α-granule secretion as measured by the increase in P-selectin on the surface of human platelets in response to agonist stimulation exhibited by a compound in accordance with an embodiment of the invention.

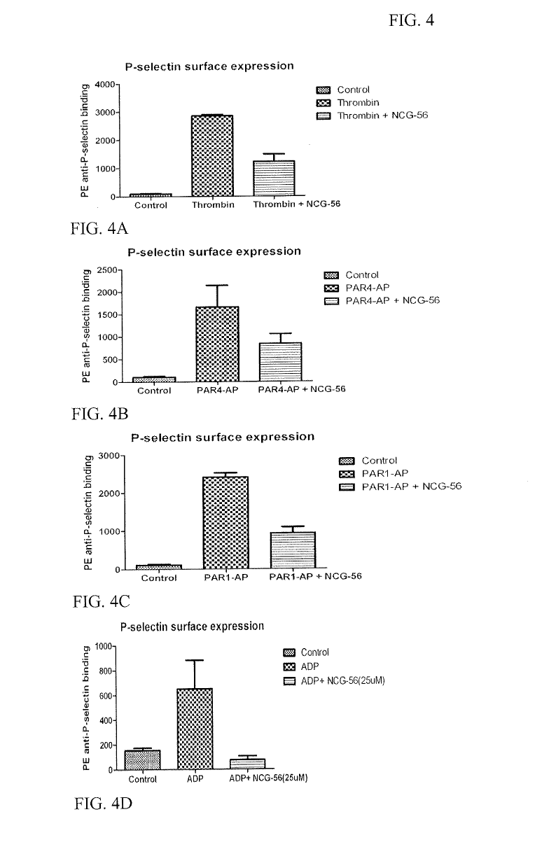

Washed platelets were obtained as described in Example 10. Flow cytometry was used to measure the secretion of α-granules. Specifically, P-selectin expression was used to detect α-granule secretion. For these experiments, 50 μl aliquots of washed platelets adjusted to a final concentration of 5×10⁵ platelets/ml were pre-treated with compound 1 for 10 min. After addition of 10 μl of PE-conjugated anti-P-selectin antibody, platelets were stimulated by the agonists thrombin, PAR1-AP, PAR4-AP or ADP for 10 min and then diluted to a final volume of 500 μl using Tyrode's buffer. The fluorescence intensity of 10,000 platelets was immediately measured using a C6 Accuri flow cytometer. The results are depicted in FIG. 4.

As is apparent from the results depicted in FIG. 4, treatment of human platelets with each of the agonists thrombin, PAR1-AP, PAR4-AP and ADP resulted in increased expression of P-selectin on the surface of the platelets as determined by the binding of anti-P-selectin. Pre-treatment of the platelets with compound 1 (identified as NCG-56 in FIG. 4) resulted in decreased expression of P-selectin on the surface of the platelets as compared to treatment with the agonists alone.

### Example 14

This example demonstrates the effect on α-granule secretion as measured by the activation of integrin αIIbβ3 in human platelets in response to agonist stimulation exhibited by a compound in accordance with an embodiment of the invention.

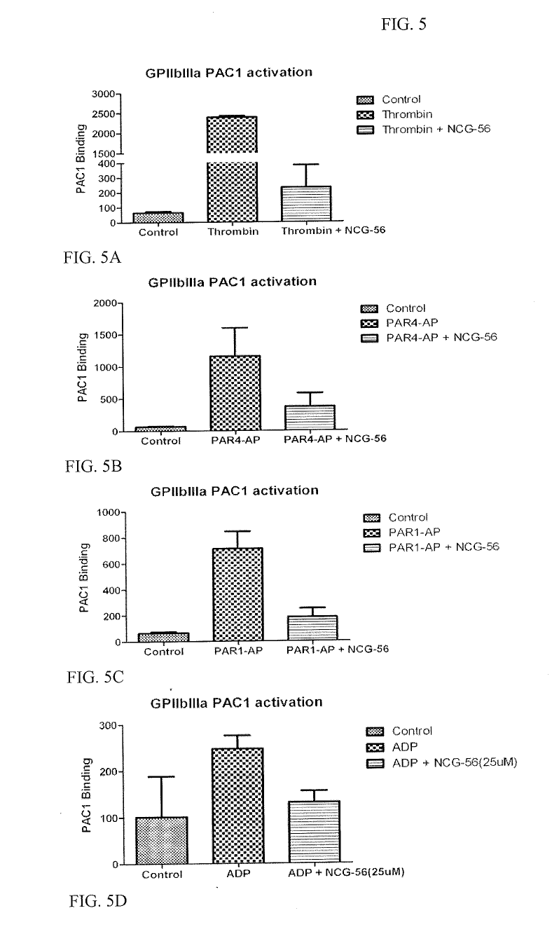

Washed platelets were obtained as described in Example 10. Flow cytometry was used to measure the activation of integrin αIIbβ3. Specifically, PAC1 (an antibody which only binds to αIIbβ3 when the protein is in its active conformation) binding was used to selectively detect the conformational activation of αIIbβ3. For these experiments, 50 μl aliquots of washed platelets adjusted to a final concentration of 5×10⁵ platelets/ml were pre treated with inhibitors for 10 min. After addition of 10 μl of FITC-conjugated PAC1, platelets were stimulated by the agonists thrombin, PAR1-AP, PAR4-AP or ADP for 10 min and then diluted to a final volume of 500 μl using Tyrode's buffer. The fluorescence intensity of 10,000 platelets was immediately measured using a C6 Accuri flow cytometer. The results are depicted in FIG. 5.

As is apparent from the results depicted in FIG. 5, treatment of human platelets with each of the agonists thrombin, PAR1-AP, PAR4-AP and ADP resulted in increased binding of PAC1 on the surface of the platelets. Pre-treatment of the platelets with compound 1 (identified as NCG-56 in FIG. 5) resulted in decreased binding of PAC1 on the surface of the platelets as compared to treatment with the agonists alone.

### Example 15

Human islets were incubated for 22 hours following stimulation with the inflammatory cytokines IFN-γ, TNF-α, and IL-1-β alone or in the presence of compound 1 or compound 9. Control islets were incubated for 22 hours in the absence of inflammatory cytokines or compounds. Following the incubation, gene expression for 12-LO, 15-LO, 5-LO, IL-12p40, and IFN-γ were determined using Taqman real time PCR. The change in gene expression relative to the control islets is set forth in Table 5.

TABLE 5
Change in Gene Expression for 12-LO, 15-LO, 5-LO, IL-
12p40, and IFN-γ in Presence of Compounds 1 and 9

| Compound | 12-LO | 15-LO | 5-LO | IL-12p40 | IFN-γ |
|---|---|---|---|---|---|
| none | 8 | 4 | 1 | 120 | 60 |
| 1 | 13 | 1 | 1 | 3 | 25 |
| 9 | 12 | 4 | 1 | 3 | 4 |

As is apparent from the data set forth in Table 4, the presence of compounds 1 and 9 resulted in a 40-fold reduction in IL-12p40 mRNA expression, and a 4-fold reduction in IFN-γ expression induced by IFN-γ, TNF-α, and IL-1-β.

### Example 16

This example illustrates the effect of 12(S)-HETE on IL-12p40 mRNA levels in human islets.

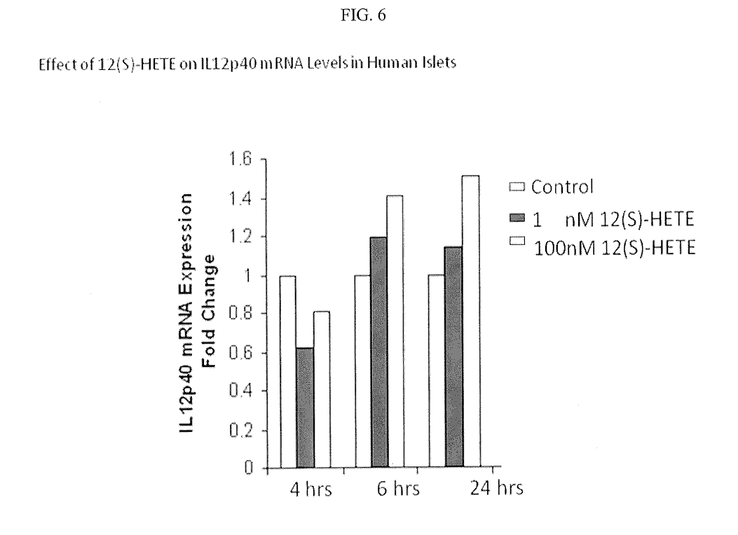

12(S)-HETE was added to cultured human islets. A control was determined by culturing islets in the absence of 12(S)-HETE. The islets were extracted for mRNA at 4 h, 6 h, and 24 h time periods and Taqman real time PCR used to determine the change in IL-12p40 mRNA expression relative to the control islets. The results are depicted in FIG. 6. In FIG. 6, at each time point, the bar on the left represents IL-12p40 mRNA expression of the control islets, the middle bar represents IL-12p40 mRNA expression of islets treated with 1 nM of 12(S)-HETE, and bar on the right represents IL-12p40 mRNA expression of islets treated with 100 nM of 12(S)-HETE.

As is apparent from the data depicted in FIG. 6, treatment of human islets with 1 nM 12(S)-HETE resulted in an approximately 1.2-fold increase in expression of IL-12p40 mRNA at both 6 h and 24 h. Treatment of human islets with 100 nM 12(S)-HETE resulted in an approximately 1.4-fold and approximately 1.5-fold increase in expression of IL-12p40 mRNA at 6 h and 24 h.

### Example 17

This example illustrates the effect of 12(S)-HETE on IFN-γ mRNA levels in human islets.

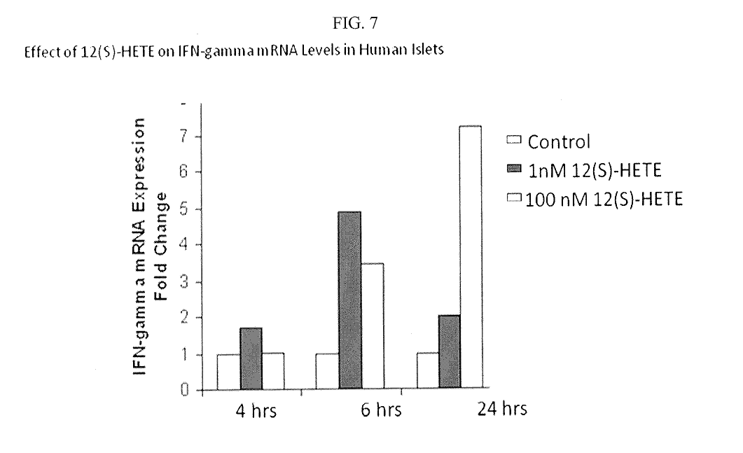

12(S)-HETE was added to cultured human islets. A control was determined by culturing islets in the absence of 12(S)-HETE. The islets were extracted for mRNA at 4 h, 6 h, and 24 h time periods and Taqman real time PCR used to determine the change in IFN-γ mRNA expression relative to the control islets. The results are depicted in FIG. 7. In FIG. 7, at each time point, the bar on the left represents IFN-γ mRNA expression of the control islets, the middle bar represents IFN-γ mRNA expression of islets treated with 1 nM of 12(S)-HETE, and bar on the right represents IFN-γ mRNA expression of islets treated with 100 nM of 12(S)-HETE.

As is apparent from the data depicted in FIG. 7, treatment of human islets with 1 nM 12(S)-HETE resulted in an approximately 2-fold, 4-fold, and 8-fold increase in expression of IFN-γ mRNA at 4 h, 6 h, and 24 h. Treatment of human islets with 100 nM 12(S)-HETE resulted in an approximately 2-fold, 3-fold, and 6-fold increase in expression of IFN-γ mRNA at 4 h, 6 h, and 24 h.

### Example 18

This example illustrates the functional bioactivity of an inventive compound of Formula II, in accordance with an embodiment, using the human 12-lipoxygenase inhibition (“12hLO”) assay.

The enzyme activity of 12hLO was determined as described in Example 3. N-((5-chloro-8-methoxyquinolin-7-yl)(furan-2-yl)methyl)propionamide (33) (comparative), N-((4-Chloro-1-hydroxynaphthalen-2-yl)(furan-2-yl)methyl)acetamide (34) (comparative), and N-((5-chloro-8-hydroxy-1,2,3,4-tetrahydroquinolin-7-yl)(furan-2-yl)methyl)propionamide (35) (invention) were screened against human 12-lipoxygenase (“12hLO”). The IC₅₀ values are set forth in Table 6.

TABLE 6
12hLO Inhibition by Representative Embodiment

| | IC₅₀ (μM) [+/−standard |
|---|---|
| Compound | deviation (μM)] |
| 33 | >75 |
| 34 | >75 |
| 35 | 3.0 [0.7] |

As is apparent from the results set forth in Table 6, compound 35 exhibited an IC₅₀ of 3.0 M against human 12-lipoxygenase.

### Example 19

This example illustrates in vitro ADME properties of a compound in accordance with an embodiment of the invention, compound 36.

TABLE 7
In vitro ADME properties for compound 36.

| aqueous kinetic solubility | Caco-2 (Pₐₚₚ 10⁻⁶ m/s | efflux ratio |
|---|---|---|
| (PBS @ pH 7.4) | @ pH 7.4) | (B→A)/(A→B) |
| 14.5 μM | 8.8 | 2.3 |

| | PBS-pH 7.4 | Mouse plasma |
|---|---|---|
| mouse liver microsome | stability: % | stability: % |
| stability (T_{1/2}) | remaining after 48 h | remaining after 48 h |
| <10 min | 100 | 98.3 |

As is apparent from the results set forth in Table 7, compound 36 exhibited acceptable aqueous kinetic solubility, good cell permeability, and excellent stability in PBS buffer and mouse plasma.

### Example 20

This example illustrates in vivo pharmacokinetic (“PK”) properties of a compound in accordance with an embodiment of the invention, compound 36.

TABLE 8
In vivo PK properties for compound 36^{a}.

| t_{1/2} (h) (plasma) | t_{1/2} (h) (brain) | [brain/plasma]^{b} | Cₘₐₓ (μM) [plasma] |
|---|---|---|---|
| 3.5 | 1.7 | 0.01 | 288 |

| Cₘₐₓ (μM) [brain] | tₘₐₓ (h) [plasma] | tₘₐₓ (h) [brain] | cLogP |
|---|---|---|---|
| 5 | 0.25 | 0.5 | 2.8 |

^{a}Intraperitoneal (IP) administration (30 mg/kg body weight (mpk)), CD1 mice, n = 3, monitored at 8 time points (0.25 h, 0.5, 1, 2, 4, 8, 12, 24). Compound 36 formulated as a suspension in 50% PEG 200 and 10% Cremophor EL in saline solution
^{b}Calculated based on the average [b/p] ratio over 8 time points (24 h period).

As is apparent from the results set forth in Table 8, compound 36 exhibited a reasonable plasma half life of 3.5 h and reasonable Cₘₐₓ of 288 M. The exposure level represented by the Cₘₐₓ exceeded the purified enzyme assay IC₅₀ for the full 24 h period and IC₅₀ in the platelet assay for 8 h. In addition, compound 36 does not efficiently cross the blood-brain barrier, which for the treatment of diseases such as diabetes and thrombosis is considered a desirable result as CNS-active compound could result in undesired side effects.

### Example 21

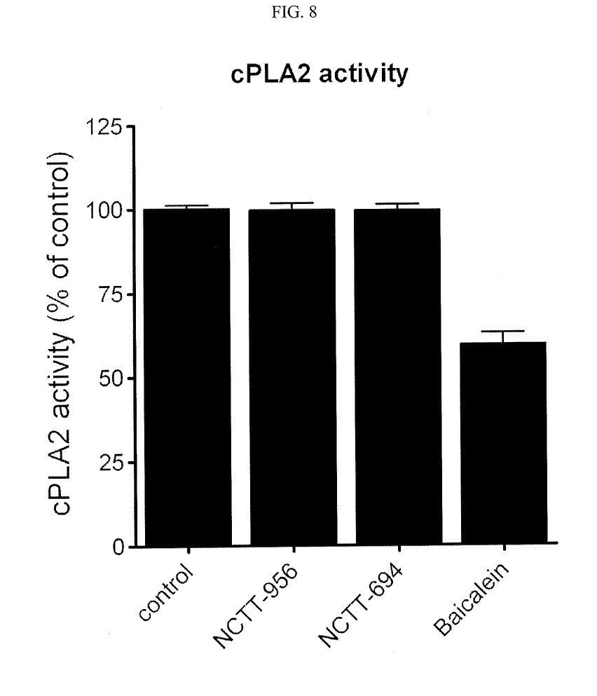

The effect of compound NCTT-956 (compound 1) (inventive), compound NCTT-694 (compound 33) (negative control), and baicalein, a nonselective inhibitor of 12hLO and 15hLO on cPLA2 (cytosolic phospholipases A2), was tested with recombinant human cPLA2 using the enzymatic activity assay described by Reed, K. A., et al., Biochemistry, 2011, 50: 1731-1738 with the following differences. The inhibitors in solution in DMSO were added at a final concentration of 50 μM right before the recombinant enzyme was added to initiate the reaction. After 5 min of incubation, the products of reaction were analyzed, and the results depicted in FIG. 8.

As is apparent from the data depicted in FIG. 8, compound 1 did not inhibit cPLA2, while the nonspecific inhibitor baicalein exhibited approximately 60% inhibition of cPLA2.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of Formula (I) or Formula (II): 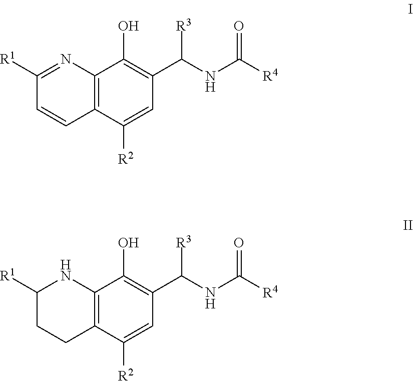
wherein R¹ and R² are independently selected from hydrogen, alkyl, alkenyl, alkynyl, aryl, heterocyclyl, heteroaryl, nitro, fluoro, bromo, chloro, and iodo,
R³ is selected from isoalkyl, cycloalkyl, aryl, heterocyclyl, and heteroaryl, each optionally substituted with one or more substituents selected from halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, heteroaryl, —NO₂, —OH, —OR⁵, —SH, —SR⁵, —SOR⁵, —SO₂R⁵, —COR⁵, —COOH, —COOR⁵, —CONHR⁵, and —CONR⁵R⁶,
R⁴ is selected from hydrogen and alkyl, wherein alkyl is optionally substituted with halo, —NO₂, —OH, —OR⁵, —SH, —SR⁵, —SOR⁵, —SO₂R⁵, —COR⁵, —COOH, —COOR⁵, —CONHR⁵, and —CONR⁵R⁶,
and
R⁵ and R⁶ are independently selected from C₁-C₆ alkyl, C₂-C₆ alkenyl, C₂-C₆ alkynyl, C₃-C₈ cycloalkyl, and C₃-C₈ cycloalkenyl,
or a pharmaceutically acceptable salt thereof, enantiomers thereof, a mixture of enantiomers thereof, or diastereomers thereof.

2. The compound, salt thereof, enantiomers thereof, a mixture of enantiomers thereof, or diastereomers thereof of claim 1, wherein the compound is of Formula (I).

3. The compound, salt thereof, enantiomers thereof, a mixture of enantiomers thereof, or diastereomers thereof of claim 2, wherein R¹ is hydrogen.

4. The compound, salt thereof, enantiomers thereof, a mixture of enantiomers thereof, or diastereomers thereof of claim 2, wherein R² is selected from nitro, fluoro, chloro, and bromo.

5. The compound, salt thereof, enantiomers thereof, a mixture of enantiomers thereof, or diastereomers thereof of claim 2, wherein R³ is selected from isoalkyl, cycloalkyl, heteroaryl, and aryl, each optionally substituted with one or more substituents selected from halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, heteroaryl, —NO₂, —OH, —OR⁵, —SH, —SR⁵, —SOR⁵, —SO₂R⁵, —COR⁵, —COOH, —COOR⁵, —CONHR⁵, and —CONR⁵R⁶.

6. The compound, salt thereof, enantiomers thereof, a mixture of enantiomers thereof, or diastereomers thereof of claim 5, wherein R³ is isoalkyl or cycloalkyl.

7. (canceled)

8. The compound, salt thereof, enantiomers thereof, a mixture of enantiomers thereof, or diastereomers thereof of claim 7, wherein the compound is selected from N-((5-chloro-8-hydroxyquinolin-7-yl)(isopropyl)methyl)propionamide, N-((5-chloro-8-hydroxyquinolin-7-yl)(isopropyl)methyl)acetamide, N-((5-chloro-8-hydroxyquinolin-7-yl)(cyclopropyl)methyl)propionamide, and N-((5-chloro-8-hydroxyquinolin-7-yl)(cyclopropyl)methyl)acetamide.

9.-11. (canceled)

12. The compound, salt thereof, enantiomers thereof, a mixture of enantiomers thereof, or diastereomers thereof of claim 5, wherein R³ is heteroaryl, optionally substituted with one or more substituents selected from halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, heteroaryl, —NO₂, —OH, —OR⁵, —SH, —SR⁵, —SOR⁵, —SO₂R⁵, —COR⁵, —COOH, —COOR⁵, —CONHR⁵, and —CONR⁵R⁶.

13. The compound, salt thereof, enantiomers thereof, a mixture of enantiomers thereof, or diastereomers thereof of claim 12, wherein R³ is selected from furan-2-yl, thiophen-2-yl, and alkylated or halogenated derivatives thereof.

14. The compound, salt thereof, enantiomers thereof, a mixture of enantiomers thereof, or diastereomers thereof of claim 13, wherein R³ is furan-2-yl or thiophen-2-yl.

15. The compound, salt thereof, enantiomers thereof, a mixture of enantiomers thereof, or diastereomers thereof of claim 14, wherein the compound is selected from N-((5-nitro-8-hydroxyquinolin-7-yl)(furan-2-yl)methyl)propionamide, N-((5-chloro-8-hydroxyquinolin-7-yl)(furan-2-yl)methyl)propionamide, N-((5-chloro-8-hydroxyquinolin-7-yl)(furan-2-yl)methyl)acetamide, N-((5-bromo-8-hydroxyquinolin-7-yl)(furan-2-yl)methyl)propionamide, N-((5-bromo-8-hydroxyquinolin-7-yl)(furan-2-yl)methyl)acetamide, N-((5-fluoro-8-hydroxyquinolin-7-yl)(furan-2-yl)methyl)acetamide, N-((5-nitro-8-hydroxyquinolin-7-yl)(thiophen-2-yl)methyl)propionamide, N-((5-chloro-8-hydroxyquinolin-7-yl)(thiophen-2-yl)methyl)propionamide, N-((5-chloro-8-hydroxyquinolin-7-yl)(thiophen-2-yl)methyl)acetamide, N-((5-bromo-8-hydroxyquinolin-7-yl)(thiophen-2-yl)methyl)propionamide, N-((5-bromo-8-hydroxyquinolin-7-yl)(thiophen-2-yl)methyl)acetamide, N-((8-hydroxyquinolin-7-yl)(thiophen-2-yl)methyl)propionamide, and N-((5-fluoro-8-hydroxyquinolin-7-yl)(thiophen-2-yl)methyl)acetamide, and N-((5-chloro-8-hydroxyquinolin-7-yl)(5-methylthiophen-2-yl)methyl)propionamide.

16.-19. (canceled)

20. The compound, salt thereof, enantiomers thereof, a mixture of enantiomers thereof, or diastereomers thereof of claim 5, wherein R³ is aryl, optionally substituted with one or more substituents selected from halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, heteroaryl, —NO₂, —OH, —OR⁵, —SH, —SR⁵, —SOR⁵, —SO₂R⁵, —COR⁵, —COOH, —COOR⁵, —CONHR⁵, and —CONR⁵R⁶.

21. The compound, salt thereof, enantiomers thereof, a mixture of enantiomers thereof, or diastereomers thereof of claim 20, wherein the compound is N-((5-chloro-8-hydroxyquinolin-7-yl)(4-methylphenyl)methyl)propionamide or N-((5-chloro-8-hydroxyquinolin-7-yl)(4-fluorophenyl)methyl)propionamide.

22.-23. (canceled)

24. The compound, salt thereof, enantiomers thereof, a mixture of enantiomers thereof, or diastereomers thereof of claim 1, wherein the compound comprises an enantiomeric excess of at least 75% of a single levorotatory enantiomer.

25. The compound, salt thereof, enantiomers thereof, a mixture of enantiomers thereof, or diastereomers thereof of claim 1, wherein the compound is of Formula (II).

26. The compound, salt thereof, enantiomers thereof, a mixture of enantiomers thereof, or diastereomers thereof of claim 25, wherein R¹ is hydrogen.

27. The compound, salt thereof, enantiomers thereof, a mixture of enantiomers thereof, or diastereomers thereof of claim 25, wherein R² is selected from nitro, fluoro, chloro, and bromo.

28. The compound, salt thereof, enantiomers thereof, a mixture of enantiomers thereof, or diastereomers thereof of claim 25, wherein R³ is selected from isoalkyl, cycloalkyl, heteroaryl, and aryl, each optionally substituted with one or more substituents selected from halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, heteroaryl, —NO₂, —OH, —OR⁵, —SH, —SR⁵, —SOR⁵, —SO₂R⁵, —COR⁵, —COOH, —COOR⁵, —CONHR⁵, and —CONR⁵R⁶.

29.-38. (canceled)

39. A pharmaceutical composition comprising the compound, salt thereof, enantiomers thereof, a mixture of enantiomers thereof, or diastereomers thereof of an, of claim 1 and a pharmaceutically acceptable carrier.

40. A method for treating or preventing a 12-lipoxygenase mediated disease or disorder, comprising administering to a mammal in need of, a therapeutically or prophylactically effective amount of a compound, salt thereof, enantiomers thereof, a mixture of enantiomers thereof, or diastereomers thereof of claim 1.

41. (canceled)

42. The method of claim 40, wherein the 12-lipoxygenase mediated disease or disorder is selected from diabetes, cardiovascular disease, and thrombosis, and myocardial infarction.

43. (canceled)

44. A method for protecting beta cells in a patient afflicted with diabetes, comprising administering to the patient a therapeutically effective amount of a compound, salt thereof, enantiomers thereof, a mixture of enantiomers thereof, or diastereomers thereof of claim 1.

45.-46. (canceled)
